SEQUENCE LISTING (5 sequences)

SEQ ID NO: 1 - DNA, 1422 bp, Homo sapiens
ggaggctgcacccggcggggcgctgatgcggcgcctggaccttcgctgcgcgacttcgggggcgtcggccgagttgggactccgcgatgcagctcctgaaggcgctctgggcactggtccacgcgcagttcctcaaagaaggtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctgacaaacacagcccctgaggggccccgggagtggccttggctccctggagagcccacgtctcagccacagttctccactcgcctcggacttcacccgttctctgccgcccgcccactccgtttccctgtggtccgtgaaggacggcctcaggccttggcatcctgagcttcggtctgtccagccgacccgaggaggccggactcagacacataggcggggggcggcacctggcatcagcaatacgcagtctgtgggagcccggccgcgcccagcccccgccgaccgtggcattggccctgctgtcctcagaggaggaggaggaggaggcagctccggcagccacagaaggctgcagcccagcccgcctgagacacgacgcctgccccaggggactgtcaggcacagaagcggcctcctcccgtgccccagactgtccgaattgcttttattttcttatactttcagtatactccatagaccaaagagcaaaatctatctgaacctggacgcaccctcactgtcagggtccctggggtcgcttgtgcgggcgggagggcaatggtggcagagacatgctggtggccccggcggagcggagagggcggccgtggtggaggcctccaccccaggagcaccccgcacaccctcggaggacgggcttcggctgcgcggaggccgtggcacacctgcgggaggcagcgacggcccccacgcagacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagcaaagaaaactcgagtaacacttgtttgaaagagatcattaaatgtattttgcaaagcctaaaaaaaaaacccacgcgtcc

SEQ ID NO: 2 - protein, 125 aa, Homo sapiens
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 3 - protein, 25 aa, Homo sapiens
QFLKEGQLAAGTCEIVTLDRDSSQP

SEQ ID NO: 4 - protein, 11 aa, Homo sapiens
TIARQTARCAC

SEQ ID NO: 5 - protein, 60 aa, Homo sapiens
GQIAGTTRARPACVDARIIKTKQWCDMLPCLEGEGCDLLINRSGWTCTQPGGRIKTTTVS

(19) United States
(12) Patent Application Publication
(10) Pub. No.: US 2009/0214514 A1
(43) Pub. Date: Aug. 27, 2009

(54) GUS3 NEUROPEPTIDES FOR REGULATING HYPOTHALAMIC FUNCTION

(76) Inventors: Leif Kongskov Larsen, Rodovre (DK); Sarag Paulsen, Charlottenlund (DE)

Correspondence Address: DARBY & DARBY P.C., P.O. BOX 770, Church Street Station, New York, NY, 10008-0770, US

(21) Appl. No.: 11/572,180

(22) PCT Filed: Jul. 21, 2005
(86) PCT No.: PCT/DK2005/000505

§ 371 Date: May 11, 2007

Related U.S. Application Data
(60) Provisional application No. 60/590,869, filed on Jul. 23, 2004.

(30) Foreign Application Priority Data
Jul. 23, 2004 (DK) PA 2004 01142

(51) Int. Cl. A61K 39/395; A61K 38/16; C12Q 1/68; G01N 33/53; A61P 3/04

(52) U.S. Cl. 424/130.1; 514/12; 435/6; 435/7.1

(57) ABSTRACT

The present invention relates to use of GUS3 neuropeptides or functional variants in a medicament. The invention furthermore relates to specific medical uses of such neuropeptides, for regulating hypothalamic function.

### TECHNICAL FIELD

The present invention relates to the field of neuropeptides and peptide hormones. More particularly, the present invention relates to neuropeptides associated with the hypothalamus, said neuropeptides being involved in regulation of homeostasis within the body of an animal.

### BACKGROUND OF THE INVENTION

The hypothalamus is involved in a large number of regulative functions. Comparative studies of the hypothalamus show that this region of the brain is anatomically, functionally, and physiologically very well conserved in vertebrates. Thus, several examples of functions as well as dysfunctions originally observed in animal models have subsequently been shown to be analogous in humans. Animal models are therefore extremely useful as a tool to gain insight into human hypothalamic functions.

Well known examples of hypothalamic dysfunctions in humans and animals are: hypothalamic hypogonadism and diabetes insipidus. Also, metabolic disorders such as obesity and accompanying diabetes mellitus and dyslipidaemia are frequently associated with abnormal function of hypothalamic neurons. Thus, several monogenetic diseases such as the metabolic syndromes associated with absent leptin synthesis (ob/ob mice), mutated leptin receptors (db/db mice, fa/fa rats), and the early onset obesity associated with melanocortin 4-receptor mutations are corrected by restoration of hypothalamic expression of the wild type gene. As a consequence, in most cases, data obtained using the hypothalamus from model animals excellently reflects human disorders involving dysfunctional hypothalamus and provides therapeutic targets for restoration of normal function.

As a central player of the limbic system, the hypothalamus is centrally placed as the overall conductor of such diverse functions as: reproduction and sexual behaviour, water and electrolyte homeostasis, energy homeostasis, blood glucose, emotions, mood, maternal behaviour, sleep and wakefulness, circadian rhythms, memory, thermoregulation, blood pressure regulation, kidney function, endocrine system (thyroid, gonadal, adrenocortical, growth, mammary function, lactation), gastrointestinal function, and immune competence.

Drugs, compounds, gene therapies, and other therapeutic devices for ameliorating, curing or modulating diseases with a hypothalamic component are suitable therapeutic tools for a number of diseases including: Hypothermia, hyperthermia, obesity, dyslipidaemia, sarcopenia, anorexia nervosa, cancer cachexia, AIDS related wasting, bulimia nervosa, diabetes mellitus, hypoglycaemia, dehydration, polyuria, electrolyte disturbances (hyponatraemia, hypernatraemia, hypokalaemia, hyperkalemia, hypocalcaemia, hypercalcaemia), diabetes insipidus, syndrome of inappropriate secretion of antidiuretic hormone (SIADH), autonomic dysfunction, arterial hypertension, arterial hypotension, (overhydration, water intoxication, sexual dysfunction, infertitility, precocious puberty, dysmenorrhea, oligomenorrhea, premature menopause, perimenopause and postmenopausal complications (including osteoporosis), hypogonadism, hyperprolactinaemia, galactorrhea, endogenous depression, stress, adrenocortical hyperfunction (Cushings disease), adrenocortical hypofunction (Addison disease), growth impairment, growth hormone insufficiency, growth hormone hypersecretion, hypothyroidism, hyperthyroidism, insomnia, Narcolepsy, somnolence, jet lag, circadian rhythm disturbances, control of melatonin mediated functions (tumour growth, reproductive function, jet lag, somnolence), inflammatory diseases, autoimmune inflammatory diseases, gastroparesis, nausea, abdominal cramps, peptic ulcers, dyspepsia, diarrhoea, and obstipation.

In particular, the hypothalamic paraventricular (PVN) and supraoptic (SON) nuclei are well known to be involved in the regulation of electrolyte and water homeostasis. In addition parvicellular subregions of the PVN are involved in metabolic regulation and thus, novel peptides and/or peptide encoding genes expressed in this region constitute targets for development of drugs for treatment of diseases related to dysfunctions in the regulation of electrolyte, water and metabolic homeostasis as well as dysfunctions in other PVN-related regulations. No drugs targeting hypothalamic subregions are currently available on the market and there is therefore a need in the art for identifying such targets for future drug development. Drugs developed to hit such targets are expected to have a major impact on the systems governing bodily homeostasis. Drugs that affect targets with a spatially limited pattern of expression are expected to exhibit a therapeutic potential with a high degree of specificity and efficacy and with very few adverse effects.

The hypothalamus is organized as a collection of distinct autonomously active nuclei with discrete functions. The hypothalamus governs several physiological variables regulated around an adjustable set point, including body composition and body temperature.

The hypothalamus is a heterogeneously paired brain structure located below the thalamus on each side of the third ventricle. The heterogeniety of the hypothalamus is well recognized, and is evident when microscopically examining this structure in Nissl stained (Cresyl violet/Thionin) sections of the mammalian brain. Based on Nissl stained material, groups or clusters of more or less densely packed neurons can be recognized. More densely packed groups of neurons are classically termed “nuclei”, whereas areas with more loosely packed neurons are termed “areas” or “zones” [1,2]. An example of a “nucleus” and an “area/zone” is given in FIG. 1A.

FIG. 1A shows a Nissl stained (thionin) section through the rat hypothalamus corresponding to Plate 26 in the atlas by Swanson [1]. All nomenclature and abbreviations for hypothalamic and extrahypothalamic nuclei and areas used herein corresponds to the nomenclature used in the brain atlas by Swanson [1]. Different nomenclatures are sometimes used in the literature in addition to the nomenclature suggested in Swanson.

In addition, the lateral hypothalamic area (LHA; Plate 22-33 [1]) has been further subdivided according to Geeraedts and co-workers [3,4]. The hypothalamic paraventricular nucleus (PVH) is depicted in FIG. 1A and from the figure (as well as from the Atlas) it can be seen that the PVH can be further sub-divided into so-called sub-nuclei; e.g. the dorsal parvicellular subnucleus (dpPVH), the posterior magnocellular subnucleus (pmlPVH) and the dorsal medial parvicellular subnucleus (mpdPVH).

On FIG. 1A—below the PVH—the SBPV (=subparaventricular zone) is depicted. This being an example of a “zone” (or “area”—that is a more loose collection of neurons). Hypothalamic “nuclei” and “areas” that are of importance in appetite and body-weight regulation include the following: the PVH, the hypothalamic arcuate nucleus (ARH; plate 26-30); the ventromedial hypothalamic nucleus (VMH, plate 26-30), the hypothalamic dorsomedial nucleus (DMH, plate 28-31), the lateral hypothalamic area (LHA, plate 22-33); the median eminence (Me, plate 26-30); the periventricular nucleus (PV, plate 19-31), the subparaventricular zone (SBPV).

As very little is known about what genes are involved in regulation of body homeostasis it is of great scientific and therapeutical interest to gain further insight into these putative neuropeptides and it is of particular interest to elucidate their specific functions. Elucidation of the function(-s) of a peptide is the first step toward generating new drugs designed to modulate hypothalamic functions and/or to cure or ameliorate hypothalamic dysfunctions.

Hardly any therapeutic targets involved in regulation of hypothalamic functions have thus far been identified. There is therefore a need in the art for obtaining tools for regulating hypothalamic functions. Such tools, directed against the prime centre for regulation of bodily homeostasis will be expected to exhibit high specificity, high efficacy, and a relatively low incidence of adverse effects.

### SUMMARY OF THE INVENTION

The present invention relates to use in a medicament of GUS3 peptides, analogues, and modulators thereof. The present invention further relates to pharmaceutical compositions, methods of treatments as well as methods of identifying GUS3 interaction partners.

### DETAILED DISCLOSURE OF THE INVENTION

The present invention relates to use in a medicament of at least one compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; a functional variant of any of these peptides; a modulator of any of these peptides; a peptide that binds polyclonal antibodies raised against any of these peptides; a DNA sequence encoding any of these peptides; and an antisense-polynucleotide to a nucleotide sequence encoding any of these peptides. The present invention further relates to use of antibodies specific to such peptides. These compounds can furthermore be used in a process for manufacturing a medicament. Methods of treatment using such compounds are furthermore contemplated. In one embodiment the compound is selected from the group consisting of a peptide with the sequence defined in SEQ ID NO 2, a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides. In another embodiment the compound is selected from the group consisting of a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides. In another embodiment the compound is GUS3C or a functional variant thereof.

Medicaments according to the present invention are useful in modulation, or regulation, of the following conditions: thirst, appetite, water and/or solute balance. In one embodiment the invention relates to use of a compound selected from a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; or a functional variant of any of these peptides, in a medicament for reduction of body weight. In another embodiment the invention relates to use of a compound selected from a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; or a functional variant of any of these peptides, in a medicament for reduction of body weight. In another embodiment the invention relates to use of a compound selected from a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; or a functional variant of any of these peptides, in a medicament for reduction of appetite or induction of satiety. In another embodiment the invention relates to use of a compound selected from a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; or a functional variant of any of these peptides, in a medicament for reduction of appetite or induction of satiety. In another embodiment the invention relates to the use of GUS3C for reduction of body weight. In another embodiment the invention relates to the use of GUS3C for reduction of appetite or induction of satiety. In another embodiment the invention relates to the use of GUS3C in a medicament for the lowering food or water intake.

Medicaments according to the present invention are furthermore useful for preventing, treating, or regulating a hypothalamic function and/or disorder in an animal.

The present invention also relates to methods for preventing, treating or regulating a hypothalamic function and/or disorder in an animal, comprising administering to the animal an effective amount of at least one active compound selected from: SEQ ID NO 2; residues 66-125 from SEQ ID NO 2; residues 53-63 from SEQ ID NO 2; residues 26-50 from SEQ ID NO 2; a functional variant of any of these peptides; a peptide that binds polyclonal antibodies raised against any of these peptides; antibodies specific to at least one of these peptides, a DNA sequence encoding any of these peptides; and an anti-sense nucleotide to a nucleotide sequence encoding any of these peptides. Examples of hypothalamic conditions include: malfunctions in water and electrolyte homeostasis; energy homeostasis; insulin resistance; dyslipidaemia; arterial blood pressure regulation; dysfunction of thirst regulation; and dysfunction of appetite regulation, a dysfunction that leads to an abnormal body weight regulation, eventually leading to type II diabetes and the metabolic syndrome X.

In one embodiment the methods for preventing, treating or regulating a hypothalamic function and/or disorder in an animal, comprising administering to the animal an effective amount of at least one active compound selected from: Residues 66-125 from SEQ ID NO 2; residues 53-63 from SEQ ID NO 2; residues 26-50 from SEQ ID NO 2; and a functional variant of any of these peptides. In one embodiment the methods for preventing, treating or regulating a hypothalamic function and/or disorder in an animal, comprises administering to the animal an effective amount of GUS3C or a functional variant thereof.

The present invention also relates to pharmaceutical compositions comprising compounds according to the invention as well as pharmaceutically acceptable ingredients. A pharmaceutical composition may also comprise siRNA polynucleotides specific for compounds according to the invention.

The present invention also relates to a method of identifying an interaction partner to GUS3 comprising using at least one compound according to the invention to screen an expression library for interaction partners. A method of identifying a modulator of GUS3 comprising use of these compounds for screening an array of compounds for binding partners and subsequently determining the effect of this binding upon the biological activity of GUS3.

Finally, the present invention relates to a method of diagnosing or prognosticating a metabolic disorder in an animal comprising determining the sequence of the polynucleotide, which encodes GUS3, and comparing the sequence with SEQ ID NO: 1 to identify differences in the sequence and using this information for diagnostic and/or prognostic purposes. Methods of determining the level of GUS3 in a biological sample using an antibody of claim 2, and using the measurement to evaluate the state of the animal are likewise contemplated.

Previous studies has revealed that the GUS3 mRNA (data base accession numbers AY358847, AAP92410 and AAP92416) encode a peptide with the basal characteristics of neuropeptides [5]. There are however, no disclosures of any specific biological roles of GUS3.

The examples below show that the GUS3 mRNA is modulated in specific areas of the hypothalamus known to be involved in regulation of water, solute, and/or metabolic homeostasis and that GUS3 is involved in regulation of thirst and food intake.

It is thus an important object of the present invention to provide tools for identification of compounds that function as modulators of mammalian water, solute, and/or metabolic homeostasis. Such compounds can be administered to a patient experiencing abnormal fluctuations in body water and solute content, either alone or as part of an adverse medical condition such as oedemas, congestive heart failure etc., for the treatment thereof as well as for the treatment of patients experiencing abnormal metabolic homeostasis, leading to obesity, type 2 diabetes and the metabolic syndrome X etc.

### DEFINITIONS AND EXPLANATIONS

“Neuropeptide” shall be understood as proteinaceous molecules made in the brain. Neuropeptides may function as e.g. neurotransmitters or hormones. The peptides might be released by neurons as intercellular messengers.

“Peptide hormones” shall be understood as chemical substances (peptides) having a specific regulatory effect on the activity of a certain organ or organs. The substances are secreted to and transported via the bloodstream to the target organs. The term “peptide hormones” includes substances that may or may not be produced by the endocrine glands.

“GUS3” refers to polypeptide products that can be derived from SEQ ID NO 2 (e.g. GUS3, GUS3N, GUS3C, and GUS3M; see Example 2). The term “mature protein” or “mature polypeptide” particularly refers to the GUS3 gene product with the signal sequence (or a fusion protein partner) removed. GUS3 polypeptides include functional variants. GUS3 polypeptides and functional variants thereof can be prepared synthetically, e.g. using well known techniques such as solid phase or solution phase peptide synthesis. Alternatively, GUS3 polypeptides can be prepared using well known genetic engineering techniques. GUS3 polypeptides can also be purified, e.g. by immunoaffinity purification, from a biological fluid, such as but not limited to plasma, serum, or urine, preferably human plasma, serum, or urine, preferably from a subject who overexpresses the polypeptide.

A “variant” of a GUS3 polynucleotide sequence means any naturally occurring or synthetic mutant of the sequence including allelic variants, degenerative variants, isoforms, sequences encoding GUS3 polypeptides and variants thereof comprising nucleotide substitutions, insertions, deletions and truncations, and derivatives of the sequence, including derivates containing chemical modifications.

“Functional variants of GUS3 polypeptides” shall in the present context be understood as variants of GUS3 and/or fragment of GUS3 with an essentially similar biological activity as wild type GUS3 (SEQ ID NO 1). A variant is a functional variant of GUS3 if the biological activity of the variant is 50% or more of the GUS3 activity, preferably 60% or more, more preferably 70% or more, even more preferably 80% or more, and most preferably 90% or more. Functional variants are peptides with a length of from 8, 10, 12, 15, 17, 20, 22, 25, 27, 30, 32, 35, 37, 40, 42, 45, 47, 50, 55, 60, 65, 70, 75, 80, 85, 90, 95, 100, 105, 110, 115, to 120 amino acids. Functional variants have a sequence identity with SEQ ID NO 2, of 80%, or more, more preferably 90% or more and even more preferably 95% or more. If the functional variant is a fragment of the full length GUS3 sequence, then the sequence identity should be calculated on basis of the variant sequence and the fragment of the wild type GUS3 sequence that corresponds to the variant sequence. Functional variants with a sequence that deviates from the wild type GUS3 sequence are preferably conserved in the domains defined as conserved (preferably no amino acid substitutions/insertions/deletions—if deviations are present then they consist primarily of conservative deviations) in FIG. 3. Deviations from the GUS3 wild type sequence are preferably located in domains that are less conserved (FIG. 3).

A functional variant is preferably a serologically compatible variant that cross-reacts with polyclonal antisera raised against wild type GUS3 peptides. The antibodies raised against GUS3 polypeptides have a capacity to bind to a serologically compatible variant of 30% or more as compared to the binding capacity of the immunogen, preferably 40% or more, even more preferably 50% or more and most preferably 60% or more.

The specific functional activity(-ies) of the GUS3 polypeptide can be tested in a transgenic mouse model. The GUS3 gene can be used in complementation studies employing transgenic mice. Transgenic vectors, including viral vectors, or cosmid clones (or phage clones) corresponding to the wild-type locus of candidate gene, can be constructed using the isolated GUS3 gene. Alternatively, GUS3 genes can be tested by examining their phenotypic effect when expressed in antisense or sense orientation in wild-type animals.

The secondary and tertiary structures of GUS3 polypeptides can be analysed by various methods known in the art. A hydrophilicity profile can be used to identify the hydrophobic and hydrophilic regions of the GUS3 polypeptide, which may indicate regions buried in the interior of the folded polypeptide, and regions accessible on the exterior of the polypeptide. In addition, secondary structural analysis can also be done, to identify regions of GUS3 polypeptide that assume specific secondary structures. Manipulation of the predicted or determined structure, including secondary structure prediction, can be accomplished using computer software programs available in the art. The GUS3 peptide sequence may be analysed by programs which predict cleavage of signal peptide to release mature peptide (see Example 1). Analogues of GUS3 polypeptide can be tested to determine whether they cross-react with antibodies specific for native GUS3 polypeptide, or specific fragments thereof. The degree of cross-reactivity provides information about structural homology or similarity of proteins, or about the accessibility of regions corresponding to portions of the polypeptide that were used to generate fragment-specific antibodies.

GUS3 polypeptides may be derivatized by the attachment of one or more chemical moieties to the protein moiety. The chemically modified derivatives may be further formulated for intraarterial, intraperitoneal, intramuscular, subcutaneous, intravenous, oral, nasal, rectal, buccal, sublingual, pulmonary, topical, transdermal, or other routes of administration. Chemical modification of biologically active proteins has been found to provide additional advantages under certain circumstances, such as increasing the stability and circulation time of the therapeutic protein and decreasing immunogenicity (See e.g. U.S. Pat. No. 4,179,337). GUS3 peptides may also be derivatized with a number of chemical moieties as exemplified in e.g. international patent application WO 02/098441, pp 15-18 which is hereby incorporated by reference.

The term “receptor” here is used in its broadest form as any protein that is further activated by GUS3 binding/interaction. As GUS3 contains a putative signal peptide and dibasic sites prone to act as posttranslational processing sites, it is conceivable that GUS3 peptide(s) might function as hormone(s) and/or neuropeptides(s), a notion further strengthened by the preferential expression in magnocellular as well as in parvocellular cells in the periventricular nucleus of the hypothalamus as well as in pancreatic beta-cells. Once the GUS3 receptor is identified, any screening technique known in the art can be used to screen for GUS3 receptor agonists or antagonists.

It is conceivable that the GUS3 receptor is located in the hypothalamus amongst other tissues. cDNA libraries from the hypothalamus as well as from other tissues can be constructed in standard expression cloning vectors. These cDNA clones might be introduced into COS cells as pools and the resulting transformants would be screened with active ligand to identify COS cells expressing the GUS3 receptor. Positive clones can then be isolated so as to recover the cloned receptor. The cloned receptor can be used in conjunction with the GUS3 ligand (assuming it is a hormone) to develop the necessary components for screening of small molecules binding to the receptor.

Knowledge of the primary sequence of the receptor, and the similarity of that sequence with proteins of known function, can provide an initial clue as to the agonists or antagonists of the protein. Identification and screening of antagonists is further facilitated by determining structural features of the protein, e.g. using X-ray crystallography, neutron diffraction, nuclear magnetic resonance spectrometry, and other techniques for structure determination. These techniques provide for the rational design or identification of agonists and antagonists. Receptor secondary and tertiary structures can be analyzed as described above in connection with GUS3 peptides.

Identification and isolation of a gene encoding a GUS3 receptor provides for expression of the receptor in quantities greater than can be isolated from natural sources, or in indicator cells that are specially engineered to indicate the activity of a receptor expressed after transfection or transformation of the cells. Accordingly, in addition to rational design of agonists and antagonists based on the structure of the GUS3 polypeptide alternative method for identifying specific ligands of the GUS3 receptor using various screening assays are known in the art.

The structure of the GUS3 receptor can be analysed by various methods known in the art. Preferably, the structure of the various domains, particularly the GUS3-binding site, is analysed. Structural analysis can be performed by identifying sequence similarity with other known proteins, particular hormone and protein receptors. The degree of similarity (or homology) can provide a basis for predicting structure and function of the GUS3 receptor, or a domain thereof. Sequence comparisons can be performed with sequences found in GenBank, using, for example, the FASTA and FASTP programs [12].

“Screening”. Synthetic libraries such as those described in a recent review [6] can be used to screen for GUS3 receptor ligands. With such libraries, receptor antagonists can be detected using cells that express the receptor without actually cloning the GUS3 receptor. Various screening techniques are known in the art for screening for analogs of polypeptides. Various libraries of chemicals are available, e.g. libraries of synthetic compounds generated over years of research, libraries of natural compounds, and combinatorial libraries, as described in greater detail, infra, for analogs of GUS3 polypeptide. Libraries may be screened for compounds that bind to anti-GUS3 polypeptide antibodies. “Phage-display technologies” can be used to isolate peptides, which bind GUS3 antibodies. A two-hybrid screening system can be used to identify proteins and other peptides, which interact with the GUS3 peptide. These techniques are well known in the art. A further assay is known as a “cis/trans” assay and is described in detail in U.S. Pat. No. 4,981,784 and WO 88/03168, for which purpose the artisan is referred.

Alternatively, assays for binding of soluble ligand to cells that express recombinant forms of the GUS3 receptor ligand-binding domain can be performed. The soluble ligands can be provided readily as recombinant or synthetic GUS3 polypeptide. The screening can be performed with recombinant cells that express the GUS3 receptor, or alternatively, using purified receptor protein, e.g. produced recombinantly as described above. For example, the ability of labelled, soluble or solubilised GUS3 receptor that includes the ligand-binding portion of the molecule can be used to screen libraries.

The term “agonist” used herein means any compound that binds to the GUS3 receptor and activates it, hereby eliciting a physiological response similar to the physiological response elicited by GUS3. A GUS3 agonist may be more effective than the native protein. For example, a GUS3 agonist variant may bind to a GUS3 receptor with higher affinity, or demonstrate a longer half-life in vivo, may be more efficiently transported to the compartment where the receptor resides, over the blood-brain barrier, or a combination of these characteristics. Nevertheless, GUS3 peptide agonist variants that are less effective than the native protein are also contemplated.

The term “antagonist” means any compound that binds to the GUS3 receptor and inhibits its activity, hereby inhibiting the normal physiological response elicited by GUS3.

An agonist or an antagonist may be a peptide with significant (>30%) amino acid identity with the GUS3 amino acid sequence or a fragment thereof.

“Modulator of GUS3” shall be understood as any compound that has an ability to bind GUS3 and subsequently exerting a detectable difference in the function of this protein. A compound with an ability to bind to GUS3 is potentially useful as a modulator of GUS3 activity if it is able to change the activity or the effect of the protein by 5% or more, preferably by at least 10% or more, even more preferably by at least 20% or more and most preferably by at least 50% or more.

“Antisense nucleic acids” are DNA or RNA molecules that are complementary to at least a portion of a specific mRNA molecule. In the cell, they hybridise to the mRNA, forming a double-stranded molecule. The cell does not translate an mRNA complexed in this double-stranded form and may degrade it rapidly. Therefore, antisense nucleic acids interfere with the expression of mRNA into protein. Oligomers of about fifteen nucleotides and molecules that hybridise to the AUG initiation codon will be particularly efficient, since they are easy to synthesize and are likely to pose fewer problems than larger molecules when introducing them into GUS3 peptide-producing cells.

“Antisense expression” also constitutes a method of downregulation of gene expression. An important advantage of this approach is that only a small portion of the gene need be expressed for effective inhibition of expression of the entire cognate mRNA. The antisense transgene will be placed under control of its own promoter or another promoter expressed in the correct cell type, and placed upstream of a polyA site. This transgene will be used to make transgenic mice. Alternatively, double-stranded small interfering RNA, (siRNA) may be used to inhibit expression of the GUS3 gene, either by administration of siRNA or constructs expressing siRNA in a pharmacologically acceptable form, or by the generation of transgenic mice expressing a short interfering RNA in the relevant cells.

“Ribozymes” are RNA molecules possessing the ability to specifically cleave other single-stranded RNA molecules in a manner somewhat analogous to DNA restriction endnucleases. Ribozymes were discovered from the observation that certain mRNA's have the ability to excise their own introns. By modifying the nucleotide sequence of these RNA's, researchers have been able to engineer molecules that recognize specific nucleotide sequences in an RNA molecule and cleave it. Because they are sequence-specific, only mRNA's with particular sequences are inactivated.

“siRNA's” (short interfering RNA's) are short (15-25 nt) double stranded RNA's that can be used to suppress the expression of virtually every gene. siRNA's act by a mechanism known as RNA interference via an ancient mechanism that is present in all eukaryotes except bakers yeast, see for instance [8]. siRNA's are double-stranded RNA molecules having a length of 21-23 nucleotides and bearing two 3′ overhanging ends. Such synthetic RNA molecules are detected by an enzyme complex, the RNA-induced silencing complex (RISC), which contains an endoribonuclease that uses the sequence encoded by the antisense strand to search for and find complementary mRNA that is subsequently destroyed. Efficient mRNA destruction by siRNA's involves a siRNA amplification step in which the siRNA acts as primer (by binding to mRNA) for the RNA-dependent RNA polymerase.

It will be evident for those skilled in the art that antisense RNA's, ribozymes, and siRNAs may be administered in a variety of forms, including but not limited to lipid-mediated administration of the RNA, lipid-mediated administration of a vector encoding the RNA, and virus-mediated administration of constructs encoding the RNA. Thus, inhibition of expression of the GUS3 gene, affects water, solute, and/or metabolic homeostasis.

Short oligonucleotides complementary to the coding and complementary strands of the GUS3 nucleic acid, or to non-coding regions of the GUS3 gene 5′, 3′, or internal (intronic) to the coding region are also useful as probes, as directly labelled oligonucleotide probes, or as primers for the polymerase chain reaction, for evaluating the presence of mutations in the GUS3 gene, or the level of expression of GUS3 mRNA. Preferably, the non-coding nucleic acids are derived from the human GUS3 gene.

“Antibodies”. GUS3 polypeptides can be produced recombinantly or by chemical synthesis, and may subsequently be used as an immunogen to generate antibodies that recognize the GUS3 polypeptide. Such antibodies include but are not limited to polyclonal, monoclonal, chimeric, single chain, Fab fragments, and a Fab expression library. The generation and function of antibodies has been described in greater detail in a number of publications, including WO 02/098441, (section “antibodies to the Neuronatin Polypeptide”, pp 19-24) which is hereby incorporated by reference.

The phrase “pharmaceutically acceptable” refers to molecular entities and compositions that are physiologically tolerable and do not typically produce an allergic or similarly untwanted reaction, such as gastric upset, dizziness and the like, when administered to a human. Preferably, as used herein, the term “pharmaceutically acceptable” means approved by a regulatory agency of the federal or a state government or listed in the US Pharmacopeia or other generally recognized pharmacopeia for use in animals, and more particularly in humans. The term “carrier” or “ingredient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or solution saline solutions and aqueous dextrose and glycerol solutions are preferably employed as carriers, particularly for injectable solutions.

Drugs can be administered in a variety of ways including intraarterial, intraperitoneal, intramuscular, subcutaneous, intravenous, oral, nasal, rectal, buccal, sublingual, pulmonary, topical, transdermal, or other routes of administration. Ways of administering e.g. polypeptide pharmaceuticals have been described in greater detail in WO 02/098441, pp 26-28 which is hereby incorporated by reference.

The phrase “therapeutically effective amount” is used herein to mean an amount sufficient to reduce by at least about 15%, preferably by at least 50%, more preferably by at least 90%, and most preferably prevent, a clinically significant deficit in the activity, function and response of the host. Alternatively, a therapeutically effective amount is sufficient to cause an improvement in a clinically significant condition in the host.

The expression “highly stringent conditions” in connection with polynucleotide hybridisation means 1 M Na⁺, a temperature of 65° C. and an incubation period of 24 hours.

The expression “animal” means any animal, preferably a mammal, wherein GUS3 or a variant form thereof is expressed.

“Gene therapy” is understood as a therapeutical method involving administration of nucleic acids. The vector construct used in connection with gene therapy may be a viral vector, an adenoviral vector, an adenovirus-associated viral vector, a lentivirus vector, a retroviral vector or a vacciniaviral vector. The packaging cell line may be a phage. The recombinant host cell may be a mammalian cell, preferably a human cell, a dog cell, a monkey cell, a rat cell or a mouse cell.

“Diagnostic Implications” include methods for detecting the presence of conditions and/or stimuli that impact upon abnormalities in arterial hypertension, body water, solute, and/or metabolic homeostasis, by reference to their ability to elicit the activities, which are mediated by GUS3 modulators. Modulator peptides can be used to produce antibodies to themselves by a variety of known techniques, and such antibodies could then be isolated and utilized as in tests for the presence of particular transcriptional activity in suspect target cells. Alternatively, nucleic acids can be employed in diagnosis. The diagnostic utility extends to methods for measuring the presence and extent of the modulators of GUS3 in cellular samples or biological extracts (or samples) taken from test subjects, so that both the nucleic acids (genomic DNA or mRNA) and/or the levels of protein in such test samples could be ascertained.

A diagnostic method may comprise examining a cellular sample or medium by means of an assay including an effective amount of an antagonist to a modulator protein, such as an anti-modulator antibody, preferably an affinity-purified polyclonal antibody, and more preferably a monoclonal antibody. In addition, it is preferable for the anti-modulator antibody molecules to be in the form of Fab, Fab′, F (ab′) 2 or F (v) portions or whole antibody molecules. As previously discussed, patients capable of benefiting from this method include those suffering from an adverse medical condition such as oedemas, congestive heart failure or other conditions where abnormal body water or solute homeostasis is a characteristic or factor. Methods for isolating the modulator and inducing anti-modulator antibodies and for determining and optimising the ability of anti-modulator antibodies to assist in the examination of the target cells are all well known in the art.

Also, antibodies and drugs that modulate the production or activity of the modulators and other recognition factors and/or their subunits may possess certain diagnostic applications and may for example, be utilized for the purpose of detecting and/or measuring conditions where abnormalities in water, solute, and/or metabolic homeostasis are or may be likely to develop. For example, the modulator peptides or their active fragments may be used to produce both polyclonal and monoclonal antibodies to themselves in a variety of cellular media, by well known techniques, such as the hybridoma technique utilizing, for example, fused mouse spleen lymphocytes and myeloma cells. Likewise, small molecules that mimic or antagonize the activity of the receptor recognition factors may be discovered or synthesized, and may be used in diagnostic and/or therapeutic protocols.

The expression “water, solute and/or metabolic homeostasis” as used herein comprises pathophysiological conditions in which body fluid dynamics and energy status are outside normal reference intervals with resulting clinically detectable perturbations, which upon amelioration improves the health of the subject. Clinical improvement of dys-regulated body fluid dynamics and energy status can be obtained by interfering with GUS3 function either by mimicking its action by use of an agonist or inhibiting its actions using an antagonist or alternative blocking strategies (immunoneutralisation, siRNA, etc.). Thus, GUS3 analogues comprise potential therapeutic tools in a number of clinical conditions including: Hypothermia, hyperthermia, obesity, dyslipidaemia, sarcopenia, anorexia nervosa, cancer cachexia, AIDS related wasting, bulimia nervosa, diabetes mellitus, hypoglycaemia, dehydration, polyuria, electrolyte disturbances (hyponatraemia, hypernatraemia, hypokalaemia, hyperkalemia), diabetes insipidus, inappropriate syndrome of antidiuretic hormone (SIADH), autonomic dysfunction, arterial hypertension, arterial hypotension, overhydration, water intoxication, autonomic dysfunction, gastroparesis, nausea, abdominal cramps, peptic ulcers, dyspepsia, diarrhoea, and obstipation.

Administration of recombinant GUS3 polypeptide results at least in changes in water and food intake. GUS3 polypeptide can be prepared using standard bacterial and/or mammalian expression vectors, synthetically, or purified from plasma or serum, all as stated in detail earlier herein. Alternatively, increased expression of native GUS3 polypeptide may be induced by homologous recombination techniques, as described supra.

For example reduction of GUS3 polypeptide activity (by antagonising the putative GUS3 receptor, immunoneutralisation with anti-GUS3 antibodies, antisense technologies) will enhance body fluid retention, which may be beneficial in clinical conditions characterised by functional dehydration, haemorrhage, decreased arterial mean pressure, renal dysfunction, diabetes insipidus, haemodynamic shock (sepsis, exposure to excessive heat, anaphylaxia, acute and chronic heart failure), severe burns, nocturnal enuresis, excessive vomiting, electrolyte disturbances. GUS3 antagonism is also likely to increase food intake.

In contrast, enhancement of GUS3 polypeptide action by use of a pharmacological GUS3 agonist or constitutively activating its putative receptor and intracellular signalling pathway, constitute useful therapeutic avenue in the treatment of arterial hypertension, fluid retention, oedema, electrolyte disturbances (e.g. hyponetraemia), and renal dysfunction, and is also expected to decrease food intake, thereby improving collective symptom complex epitomising the metabolic syndrome (dyslipidaemia, visceral obesity, insulin resistance, endothelial dysfunction).

### BRIEF DESCRIPTION OF THE DRAWINGS

FIG. 1: Cross-sections of hypothalamus subregions from rats. Scalebars=100 μm. A: Nissl-stained section illustrating the cytoarchitecture and the delineation of hypothalamic subregions. Dashed line shows the subdivisions of the PVH (=paraventricular nucleus of the hypothalamus). dpPVH=dorsal parvicellular subnucleus of the PVH; pmlPVH posterior magnocellular subnucleus; mpPVH medial parvicellular subnucleus of the PVH; SBPV=subparaventricular zone; 3V=third ventricle; AHN=anterior hypothalamic nucleus. B: Fluoroscence microphotograph showing retrogradely labeled neurons in the PVH. Brightest neurons (whitest; thin arrows) are labelled with True Blue injected into the dorsal vagal complex; more faintly labelled neurons (fat arrows) contain the retrograde tracer Fluorogold (rats injected intraperitoneally with Fluorogold). The two tracers are localized in different hypothalamic subregions. C: A hypothalamic subregion defined on the basis of neurotransmitter content. An antibody to oxytocin was used to label cells in the PVH containing this neuropeptide (dark neurons). D: A hypothalamic subregion defined on the basis of projection pattern. Cholera Toxin subunit B (ChB) was injected into the PVH and retrogradely labeled neurons in the DMH (neurons projecting to the PVN) are visualized using a peroxidase coupled ChB antibody and stained using diaminobenzidine as a chromogen.

FIG. 2 shows the predicted signal-peptide and cleavage site in GUS3. The output from the SignalP program employing two different modes of prediction. 1A hidden markow model prediction. 1B neural network prediction.

FIG. 3 shows an alignment of GUS3 sequences from Homo sapiens, rattus norvegicus, Mus musculus, Bos Taurus, Tetraodon nigroviridis, and Danio rerio.

FIG. 4 shows a PCR multiplex analysis of the expression of GUS3 in various tissues and cell lines.

FIG. 5 shows autoradiograms of rat brain sections through the hypothalamus hybridized with a GUS3 cRNA sense (A) and antisense probe (B).

FIG. 6 shows double in situ hybridisation of GUS3 radioactively labelled cRNA with vasopressin (A) and oxytocin (B) fluorescently labelled probes.

FIG. 7 shows GUS3 mRNA expression levels in the PVN of rats allowed free access to water, in rats salt-loaded with 1.5% NaCl for 5 days), in rats thirsted for 48 hr, and in rats thirsted for 48 hr and subsequently allowed access to water for 1 hr.

FIG. 8 shows the acute effect of 10 μg of each of the GUS3 peptides and vehicle on food (A) and water (B) intake. Animals are injected with the substances after a 24 hr thirst period and food and water intake is monitored at 30 minutes, 60 minutes, 2 hours, 3 hours, and 24 hours.

FIG. 9 shows western blotting with anti-GUS 3 antisera directed against the GUS 3 N (FIG. 9A), GUS 3 M (FIG. 9B), or GUS 3 C (FIG. 9C), respectively. Protein standards (lane S), GUS 3 peptides (GUS 3 N in FIG. 9A, GUS 3 M in FIG. 9B, and GUS 3 C in FIG. 9C, respectively), and protein extracts from hypothalamus (lane 2), pancreas (lane 3), plasma (lane 4), and cerebrospinal fluid (lane 4) are run on Criterion peptide gels (Biorad), blotted onto PVDF membrane, and finally detected by chemoluminiscense. Shown is also the location of specifically recognized polypeptides (P1, P2, and P3)

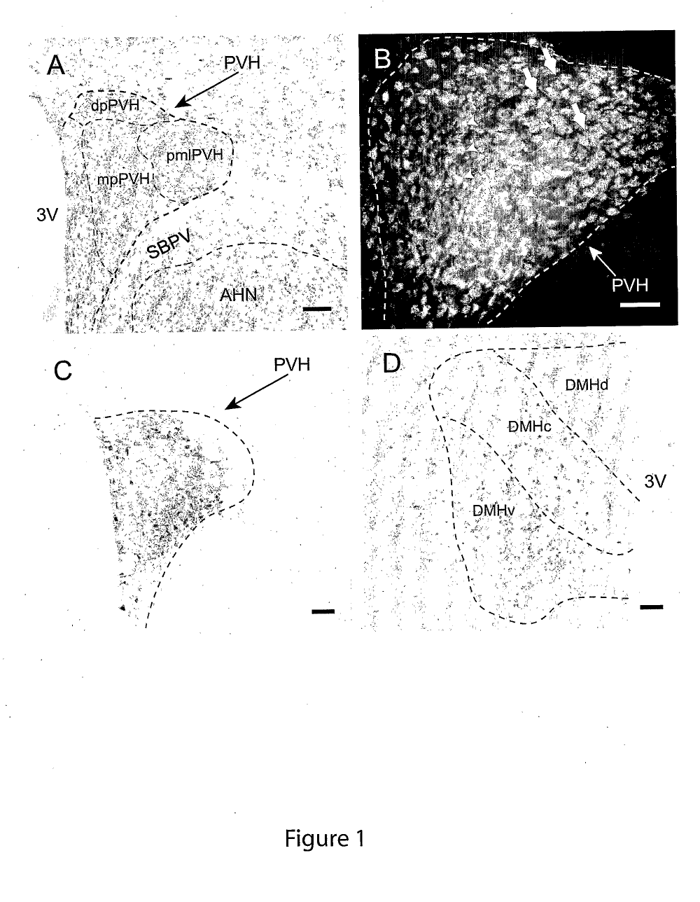

### EXAMPLES

The invention is further illustrated with reference to the following examples, which are not intended to be in any way limiting to the scope of the invention as claimed.

### Example 1

### Prediction of Signal Peptide Sequences and Cleavage Sites in GUS3

The GUS3 sequence, Genbank accession number NP_{—}598857, was analysed using the SignalP server (www.cbs.dtu.dk/services/SignalP-2.0/SignalP). The SignalP server predicts the presence and location of signal peptide cleavage sites in amino acid sequences. The method employs a prediction of cleavage sites and a signal peptide/non-signal peptide prediction based on a combination of several artificial neural networks and hidden Markov models [19].

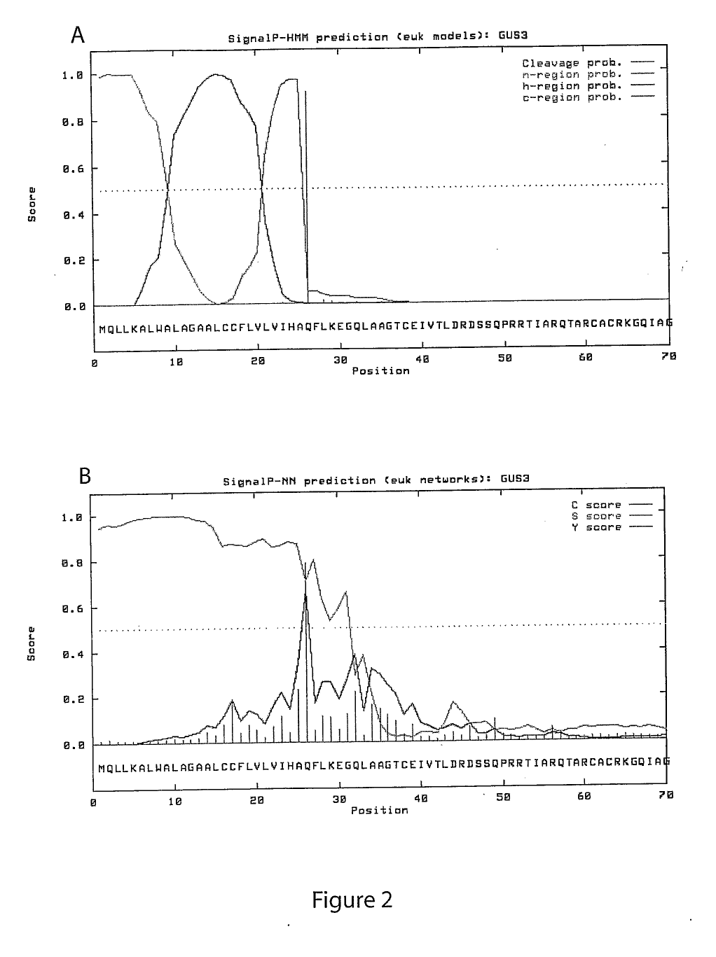

The analysis of GUS3 [A] gave the following results (see also the graphic output from the analysis in FIG. 2):

Neural networks based part:

>GUS3 length=70
# Measure Position Value Cutoff signal peptide?

| | | | | |
|---|---|---|---|---|
| max. C | 26 | 0.788 | 0.33 | YES |
| max. Y | 26 | 0.671 | 0.32 | YES |
| max. S | 9 | 0.998 | 0.82 | YES |
| mean S | 1-25 | 0.935 | 0.47 | YES |

# Most likely cleavage site between pos. 25 and 26: IHA-QF

Hidden Markov Models based part:

### >GUS3

Prediction: Signal peptide
Signal peptide probability: 0.987
Signal anchor probability: 0.013
Max cleavage site probability: 0.919 between pos. 25 and 26

This analysis predicts that native GUS3 fulfils the criteria of a pre-protein; the first 25 amino acids are predicted to have a high probability of functioning as a signal peptide. This signal peptide is predicted to be proteolytically removed post-translationally whereby a physiologically active peptide or propeptide is generated (see also example 2).

The prediction that the GUS3 peptide is a secreted peptide confirms the disclosure on the database (accession numbers AAP92410, AAP92416) where the peptide is also predicted to be a secreted protein.

### Example 2

### Evolutionary Conservation of GUS3

Homologues of the GUS3 polypeptide were found by searching with the BLAST and BLAT programs, and aligning the found polypeptide sequences or the translated genomic or cDNA sequences originating from mouse, rat, cow, zebrafish (Danio rerio), and green spotted pufferfish (Tetraodon nigroviridis) (FIG. 2) using the ClustalW algorithm [21] embedded in the DSGene program suite (Accelrys Inc.).

The GUS3 gene is amazingly highly conserved throughout evolution. Thus, the amino acid sequence of GUS3 is completely conserved from human to mouse and rat, whereas only a few substitutions are seen between mammal and fish GUS3 sequences. Most of these substitutions are conservative substitutions resulting in the substitution of amino acid residues with other amino acid recidues with similar chemical properties. The amino acid sequence from position 30 to 125 harbours e.g. 2 substitutions between Bos taurus to the other mammals.

The high degree of conservation suggests that the polypeptide has a vital and conserved function. The high degree of conservation in general and of the cysteine residues in particular indicates that the protein is similarly folded throughout evolution.

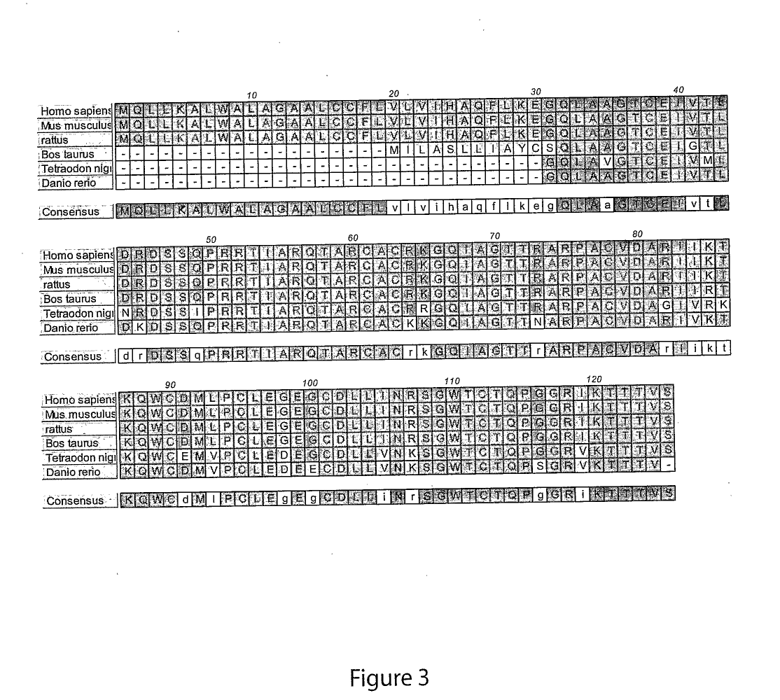

The high degree of conservation also indicates that most of the amino acid residues presented in FIG. 3 are important for correct folding and function of the polypeptide. Amino acid residues located in areas that are less conserved may be substituted or otherwise altered and still retain function of the protein.

In particular, the potential dibasic cleavage sites at position 51-52 (RR in all species) and 64-65 (RK except in T. negroviridis (RR) and D. rerio (KK)) are functionally conserved in all these evolutionary distinct species, leading credibility to the notion that the polypeptide of sequence ID NO: 2 may be processed into one or more of the following fragments:

### GUS3 N (SEQ ID NO 3):

QFLKEGQLAAGTCEIVTLDRDSSQP, positioned between the predicted N-terminal sequence peptide and the potential dibasic cleavage site closest to the N-terminal.

### GUS3 M (SEQ ID NO 4):

TIARQTARCAC, positioned between the two potential dibasic cleavage sites. This fragment is 100% conserved between all the GUS3 homologues examined (cf. FIG. 3).

### GUS3C (SEQ ID NO 5)

GQIAGTTRARPACVDARIIKTKQWCDMLPCLEGEGCDLLINRSGWTCTQPG GRIKTTTVS, positioned C-terminal to the most C-terminal potential dibasic cleavage site.

Other (larger or smaller) fragments of GUS3 may possess biological activity.

### Example 3

### Identification of GUS3 mRNA Expressing Tissues by Multiplex PCR

The procedure is based on methods described previously by Jensen et al. [22].

Fresh tissue samples from: ileum, duodenum, stomach, adrenal gland, kidney, lung, liver, hypothalamus, whole brain, heart, muscle, testis, colon, jejenum, interscapular brown adipose tissue, mesenteric white adipose tissue, epididymal white adipose tissue, perirenal white adipose tissue, inguinal white adipose tissue, and spleen were isolated from Sprague-Dawley rats and immediately submerged in RNAlater (Ambion, Tex., U.S.A.).

Total RNA was then extracted from the tissue samples and from rat pancreatic β-cell containing islets, from NHI GI28 insulinoma, and from a glucagon producing cell lines (12C3AN) using RNeasy spin columns (QIAGEN Inc., California, USA), following the manufacturer's instructions.

First-strand cDNA was prepared using 1 μg total RNA, the Superscript RT kit, and random hexamer primers (GIBCO BRL, Gaithersburg, Md., USA), according to the manufacturer's instructions. The cDNA was diluted 1:6 in distilled water. A PCR mixture was prepared. For 13.5 μl, 1.35 μl 10× polymerase buffer with MgCl₂, 0.20 μl dNTP (4 mM, 2 mM dCTP), 0.25 μl of each primer (10 mM), 0.125 μl Taq polymerase, 0.0625 μl 33P-α-dCTP (10 mCi/ml, Amersham), 1.5 μl cDNA solution, and finally water to 13.5 μl was used. Two primer sets were included in each reaction, 1 set specific for GUS3 (5′-ATGCAGCTCCTGAAGGCG-3′ and 5′-GTCCACACAAGCAGGCCG-3′, product length 240 bp), the second set specific for TBP (5′-ACCCTTCACCAATGACTCCTATG-3′ and 5′-TGACTGCAGCAAATCGCTTGG-3′, product length 186 bp) and used as an internal standard. All samples were subjected to 25 rounds of amplification in the following PCR program: An initial denaturation (2 min. 94 degrees), 25 rounds of denaturation (30 sec. 94 degrees), annealing (30 sec. 55 degrees) and elongation (30 sec. 72 degrees), and finally a long elongation period (5 min. 72 degrees).

The number of cycles was chosen in the range where the limiting factor for the amount of product is the amount of input template cDNA. The final PCR reactions were mixed with 98% formamide denaturing loading buffer and loaded in duplicate and separated on a 6% (wt/vol) polyacrylamide gel, containing 7 M urea. The gel was subsequently dried, exposed to a phosphorimager screen, and the resulting scan analyzed using Quantity One (Biorad).

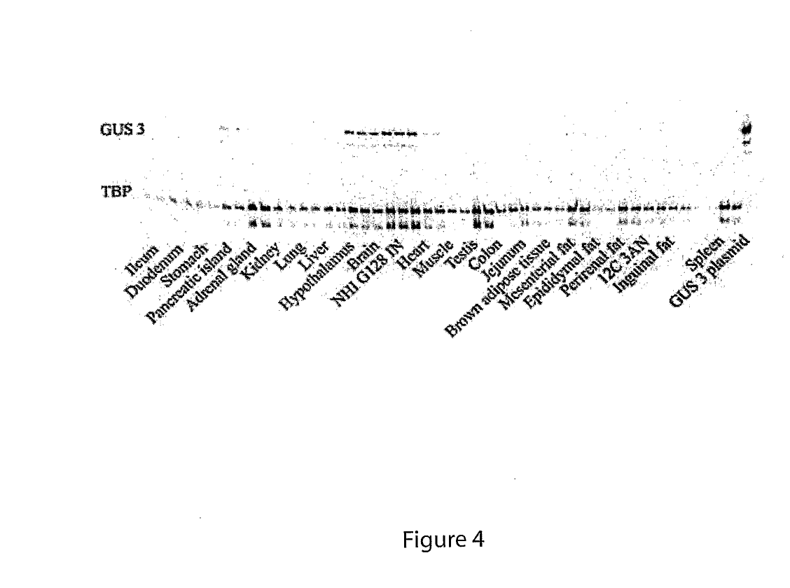

The analysis showed (FIG. 4) that the GUS 3 gene is expressed in the hypothalamus as well as in rat islets and in the insulin-producing cell line NHI G128 IN but not in the glucagons producing cell line 12C 3AN, indicating a role of GUS3 in centrally controlled homeostasis and/or as a β-cell secreted hormone.

### Example 4

### Identification of Hypothalamic Areas in the Mouse and Rat Expressing GUS3 mRNA

Cloning of a 240 Base-pair GUS3 cDNA Fragment into Plasmid Vector:

Total RNA was extracted from hypothalami obtained from male Sprague-Dawley rats (Charles River, Sweden) using the RNeasy RNA purification kit (Qiagen, Maryland, USA). Integrity and concentration of the extracted RNA was evaluated on a gel. RT-PCR was performed in a total volume of 20 μl using 1 μg total RNA, 20 U of Superscript Reverse Transcriptase [(GIBCO,] Life Technologies), enzyme buffer [(1×),] 10 mM DTT, 2 pM/μl oligodT primers, 1 mM dNTP and 0.5 μl 40 U/ul RNase inhibitor. The mixture was incubated for 1 hr at 37 degrees C. To generate a GUS3 PCR fragment, 2 μl of the template cDNA reaction was mixed with primers complementary to the rat GUS3 cDNA sequence (deduced from genomic data using the mouse GUS3 amino acid sequence with accession no: NP_{—}598857; primers: 5′-ATGCAGCTCCTGAAGGCG-3′ and 5′-GTCCACACAAGCAGGCCG-3′. The final reaction mix (total volume 50 μl contained 2 μL cDNA reaction, 1.5 mM (MgCl₂,] 0.75 mM of each primer, 1.25 U Taq DNA polymerase (Sigma-Aldrich), 1× buffer, 0.2 mM dNTP's.

The size of the generated PCR product was checked on a gel. The PCR product was cloned into pCR4-TOPO, following the manufacturers guidelines (Invitrogen, Carlsbad). Subsequently, E. coli TOP 10 cells were transfected with the plasmid DNA and grown on Ampicillin/X-gal containing culture plates. The insert of a positive clone was analyzed by PCR and the PCR-product sequenced (sequencing by MWG-biotech, Germany). The obtained raw data sequence was analyzed using the BLAST and BLAT search engines verifying a 100% identity to rat GUS3 cDNA.

In analogy, full length cDNA can be cloned from mouse, human, or rat cDNA by similar procedures using primers complementary to the full-length mouse, human, or rat cDNA sequences.

### Plasmid Purification, Linearization and In Vitro Transcription:

Plasmid containing E. coli were grown overnight at 37° C. in LB medium and plasmid DNA purified using the Qiagen Midi-Prep kit. For the in vitro transcription plasmid DNA was linearized using restriction enzymes (antisense: Not I, sense: Pme I). 33P labelled antisense (T3 RNA polymerase) and sense (T7 RNA polymerase) cRNA probes (cRNA probes are RNA probes generated by antisense transcription of cloned cDNA) were prepared as follows in a volume of 25 μl: 1× transcription buffer, 1 mM DTT, RNase inhibitor (1.6 u/ul), CTP/ATP/GTP mix (1.6 mM each), 10 μl 10 mCi/ml ³³P-ALFA-UTP (Amersham Pharmacia), linearized DNA (1 μg) and polymerase (T3 or T7, 40 u) were mixed and incubated for 2 hours at 37° C. Subsequently, the template DNA was digested by the addition of 1 μL RQ1 Dnase, 2 μL yeast tRNA and 1 μL RNase inhibitor (40 u). The transcripts were purified by phenol-chloroform extraction followed by precipitation in 2.0 M ammonium acetate and ethanol. The pellet was resuspended in 50 μl 10 mM DTT and 50 μl hydrolysing buffer (80 mM NaHCO₃, 120 mM Na₂CO₃, 10 mM DTT) and incubated at 60° C. for 53 minutes. After incubation, 100 μl neutralizing-buffer (0.2 M Na-acetat, 175 mM acetic acid, 10 mM DTT) was added and the cRNA again precipitated with ammonium acetate and ethanol. The transcripts were diluted in a 1:1 mixture of 100% de-ionized formamide and Tris (10 mM), EDTA (1 mM)-DTT (10 mM) buffer (pH 7.5). The specific activity of the generated transcripts was determined using a beta-counter.

### In Situ Hybridization:

Sprague-Dawley rats were sacrificed by decapitation and their brains removed and immediately frozen on dry ice. Twelve micron thick frontal sections were cut in a cryostat and mounted directly on Superfrost™ Plus slides. Dried slides were fixed for 5 min in 4% paraformaldehyde. The slides were next rinsed 2×5 min in phosphate buffered saline (PBS; pH 7.4) followed by a brief acetylation: 500 μL acetic anhydride (100%) was added to 200 mL 0.1 M triethanolamine and the slides immediately submerged for 2 min. Next, slides were passed through PBS twice (2×2 min) and finally through graded ethanol concentrations [(30/60/80/96/99/99)] and allowed to dry. The radioactively labelled probe was denatured for 3 min at 80° C. immediately prior to hybridisation and mixed with hybridization buffer. The hybridization buffer consisted of 50% deionised formamide, 1×SALTS (300 mM NaCl, 10 mM Tris, 10 mM NAPO₄] (pH 6.8), 5 mM EDTA, 0.02% Ficoll 400, 0.2% polyvinylpyrolidone (PVP-40, 40000 MW), 0.2% BSA Fraction V), 10% dextran sulphate, 1 μg/μL yeast tRNA and 9 mM DTT. Probe was added so that the final activity of the hybridization mix was approximately 16.000 cpm/μL. The hybridization mix was applied onto the sections (35 μL/section) that were subsequently cover-slipped. Hybridization was performed overnight at 47° C.

The next day sections were subjected to two stringency washes at 62 and 67° C. The sections were washed for 1 hour at each temperature (lowest first) in a washing buffer consisting of 50% formamide, 1×SALTS. The sections were next rinsed twice (2×2 min) in NTE buffer (0.5 M [NaCl,] 10 mM Tris-Cl (pH 7.2), 1 mM EDTA), whereafter they were RNAse A treated (20 ng/mL; Boehringer-Mannheim) for 30 min. Subsequently, the sections were rinsed twice for 5 min in NTE, 30 min in SSC (15 mM NaCl, 1.5 mM trisodiumcitrat, (pH=7.0)) and finally dehydrated through a series of graded ethanol solutions containing 0.3M ammonium acetate (30/60/80/90/99). After drying the hybridized sections were exposed to Kodak bio-max film for several days prior to development.

Localization of GUS3 mRNA in the Rat Hypothalamus:

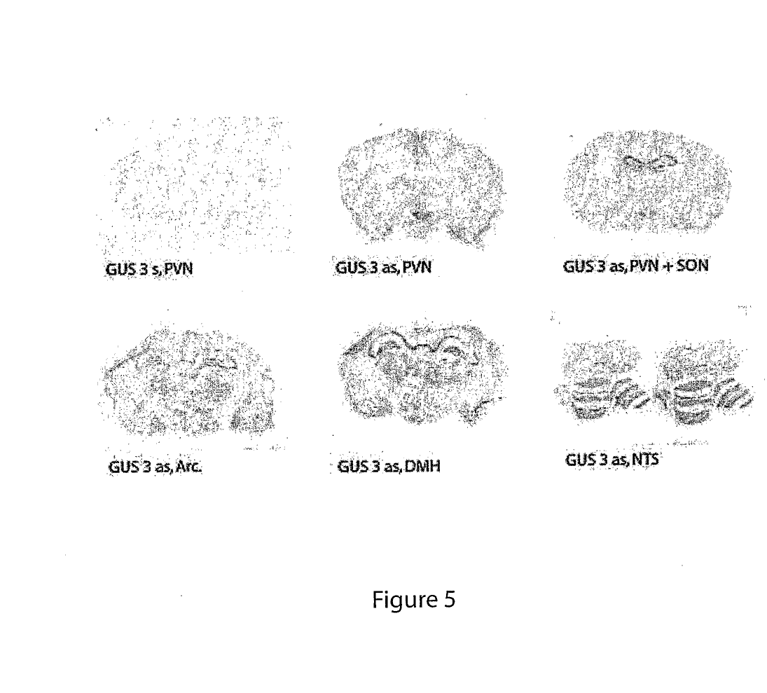

FIG. 5 shows autoradiograms of frontal hypothalamic sections from Spreague-Dawley rats. A. Sections incubated with a sense cRNA probe shows that no non-specific signal can be detected. B. From the level of the paraventricular nucleus of the hypothalamus (PVN); shows presence of GUS3 mRNA expression in this nucleus in subregions known to contain both parvocellular and magnocellular neurones. The GUS3 gene is also seen expressed in the supraoptical nucleus (SON) known to contain exclusively magnocellular neurones. Hypothalamic magnocellular neurones in the PVN and SON comprise the majority of hypothalamo-neurohypophysial system. Outside the hypothalamus expression is seen in the hippocampus.

### Example 5

### Analysis of GUS3 Expressing Cells in the PVN by Double In Situ Hybridisation

The neuroanatomical localization of GUS3 expression was determined by double in situ hybridisation using GUS3 antisense cRNA in conjunction with fluorescently labelled vasopressin and oxytocin probes. Vasopressin is a hormone that functions as a stimulator of thirst and oxytocin is a hormone that functions in regulation of appetite as well as regulation of lactation (referencer).

The PVN can be divided into eight subdivisions of which three are magnocellular and five parvocellular. The magnocellular cells are quite large cells with rather simpler dendritic trees, which can be interconnected by gap junctions. The parvocellular cells that also have simple dendritic trees, are smaller and can contact dendrites of cells in both the magnocellular and parvocellular divisions of the PVN.

One group of magnocellular neurons from the PVN and SON give rise to axons terminating within the posterior lobe of the pituitary and provide a direct neural connection between the hypothalamus and pituitary. The magnocellular neurons synthesize vasopressin and oxytocin whereas the parvocellullar neurons synthesize a large number of neurotransmitters. Neurons from parvocellular subnuclei project to all blood brain barrier free circumventricular organs as well as to hypothalamic nuclei and autonomic areas in the barin stem and the spinal cord. Double in situ hybridization experiments with Gus3 together with oxytocin and vasopressin probes are therefore important for ascertaining whether Gus3 exerts its effects by acting as a neuropeptide or as a peptide hormone

### Cloning of Vasopressin and Oxytocin Fragments

Vasopressin and oxytocin were cloned into the pCR4 top( ) vector essentially as described above for the GUS3 fragment. The cloned vasopressin fragment covers position 20 to 397 of accession number M25646, whereas the cloned oxytocin fragment covers position 3 to 237 of accession number M25649.

### Plasmid Purification, Linearization and In Vitro Transcription:

Plasmid containing E. coli were grown overnight at 37° C. in LB medium and plasmid DNA purified using the Qiagen Midi-Prep kit. For the in vitro transcription GUS3 plasmid DNA was linearized using restriction enzymes (antisense: Not I, sense: Pme I). 33P labelled antisense (T3 RNA polymerase) and sense (T7 RNA polymerase). ³³P labelled cRNA probes were prepared as follows in a volume of 25 μl: 1× transcription buffer, 1 mM DTT, RNase inhibitor (1.6 u/ul), CTP/ATP/GTP mix (1.6 mM), 10 μl 10 mCi/ml ³³P-ALFA-UTP (Amersham Pharmacia), linearized DNA (1 μg) and polymerase (T3 or T7, 40 u) were mixed and incubated for 2 hours at 37° C. Subsequently, the template DNA was digested by the addition of 1 μL RQ1 Dnase, 2 μL yeast tRNA and 1 μL RNase inhibitor (40 u). The transcripts were purified by phenol-chloroform extraction followed by precipitation in 2.0 M ammonium acetate and ethanol. The pellet was resuspended in 50 μl 10 mM DTT and 50 μl hydrolysing buffer (80 mM NaHCO₃, 120 mM Na₂CO₃, 10 mM DTT) and incubated at 60° C. for 53 minutes. After incubation, 100 μl neutralizing buffer (0.2 M Na-acetat, 175 mM acetic acid, 10 mM DTT) was added and the cRNA again precipitated with ammonium acetate and ethanol. The transcripts were diluted in a 1:1 mixture of 100% de-ionized formamide and Tris (10 mM), EDTA (1 mM)-DTT (10 mM) buffer (pH 7.5). The specific activity of the generated transcripts was determined using a beta-counter.

Dig-labeled cRNA probes were prepared as follows: 1× transcription buffer, 1 mM DTT, RNase inhibitor (0.5 u/ul), CTP/ATP/GTP mix (0.8 mM), UTP (0.5 mM), Dig-11-UTP (0.3 mM), linearized DNA (1 μg) and polymerase (T3 or T7, 40 u) were mixed and incubated for 2 hours at 37° C. Subsequently, the template DNA was digested by the addition of 1 μL RQ1 Dnase, 2 μL yeast tRNA and 1 μL RNase inhibitor (40 u). The transcripts were purified by phenol-chloroform extraction followed by precipitation in 2.0 M ammonium acetate and ethanol. The pellet was resuspended in 50 μL 10 mM DTT and 50 μL hydrolysing buffer (80 mM NaHCO₃, 120 mM Na₂CO₃, 10 mM DTT) and incubated at 60° C. for 52 (oxytoxin) or 67 (vasopressin) minutes. After incubation, 100 μL neutralizing buffer (0.2 M Na-acetat, 175 mM acetic acid, 10 mM DTT) was added and the cRNA again precipitated with ammonium acetate and ethanol. The transcripts were dissolved in 49 μL water and 1 μL Rnasin (40u).

### In Situ Hybridization:

Sprague-Dawley rats were sacrificed by decapitation and their brains removed and immediately frozen on dry ice. Twelve micron thick frontal sections were cut in a cryostat and mounted directly on Superfrost™ Plus slides. Dried slides were fixed for 5 min in 4% paraformaldehyde. The slides were next rinsed 2×5 min in phosphate buffered saline (PBS; pH 7.4) followed by a brief acetylation: 500 μL acetic anhydride (100%) was added to 200 mL 0.1M triethanolamine and the slides immediately submerged for 2 min. Next, slides were passed through PBS twice (2×2 min) and finally through graded ethanol concentrations [(30/60/80/96/99/99)] and allowed to dry. The mixtures of probes (GUS3+oxytocin and GUS3+vasopressin, respectively) were denatured for 3 min at 80° C. immediately prior to hybridisation and mixed with hybridization buffer as described above.

Probe was added so that the final activity of the radioactive probe in the hybridization mix was approximately 16.000 cpm/μL whereas the Dig-labelled probe was diluted 100-fold in the hybridization mix. The hybridization mix was applied onto the sections (35 μL/section) that were subsequently cover-slipped. Hybridization was performed overnight at 47° C. The next day sections were subjected to two stringency washes at 62 and 67° C. The sections were washed for 1 hour at each temperature (lowest first) in a washing buffer consisting of 50% formamide, 1×SALTS and 9 mM DTT. The sections were next rinsed twice (2×2 min) in NTE buffer (0.5 M NaCl, 10 mM Tris-Cl (pH 7.2), 1 mM EDTA), whereafter they were RNAse A treated (20 ng/mL; Boehringer-Mannheim) for 30 min. Subsequently, the sections were rinsed twice for 5 min in NTE, 30 min in SSC (15 mM NaCl, 1.5 mM trisodium citrate, (pH=7.0)). The slides were washed in washing buffer (4×SSC+0.1% Triton X-100) for 5 min., were blocked by incubation in blocking buffer (5% BSA in washing buffer) for 30 min., and incubated with anti-Dig antibody at 40 overnight. The slides were washed 5 times with PBST (PBS+0.25% Triton X-100), incubated with biotinylated donkey anti-sheep diluted in Blocking buffer (Fab2 fragment) 1:1000 for 1 hour, washed with PBST-buffer 5 times and incubated with ABC complex (Vector elite kit). The slides were washed 5 times with PBS-buffer and incubated in biotinylated tyramine for 13 minutes (10 ml solution contained: 10 ml PBST and 1.3 μl 35% H₂O₂ mixed with 100 μl TSA solution made by mixing 25 mg of NHS-LC-biotin and 7.8 mg of tyramine with 10 ml KPBS (0.89% NaCl, 0.02% KCl, 10 mM Phosphate buffer). The slides were washed with PBST-buffer 5 times and were incubated with Alexa Fluor 488 Streptavidin conjugate (Molecular Probes) diluted 1:200 in PBST for 45 minutes. The slides were washed with PBST 5 times and finally dehydrated through a graded ethanol series (70%, 96%, and 99%). The sections were allowed to dry and subsequently dipped in Emulsion (K5, Agfa) and allowed to expose for 16 days before development and a final thionein staining.

### Double In Situ Results

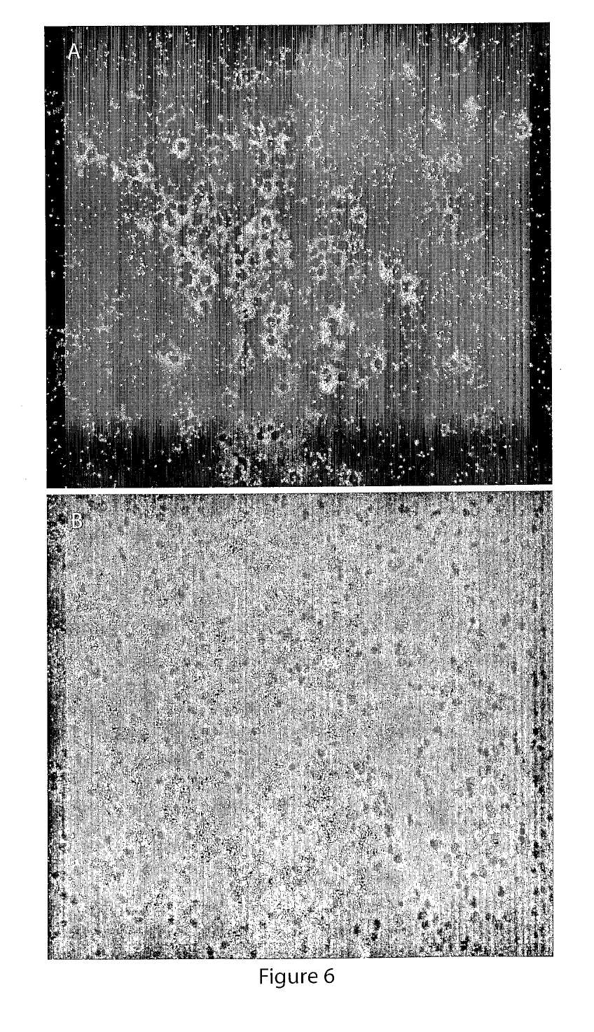

The double in situ hybridisation with GUS3 in conjunction with vasopressin or oxytocin probes show GUS3 expression in a number of neurones in the PVN (FIG. 6). Some of these neurones are double-labelled with the vasopressin probe and some of these neurones are double-labelled with the oxytocin probe, but the signal of GUS3 is clearly not limited to the magnocellular neurones.

These results leave the question of the function of Gus3 quite open. The presence in the magnocellular neurones indicate that Gus3 could function as a hormone (in the bloodstream), but a role as a neurotransmitter is also possible when considering the expression in the parvocellular neurones.

### Example 6

### GUS3 mRNA Expression Examined in a Rodent Model of Thirst and Salt-loading

Thirty male Sp. Dawley rats (250 g.) were divided into 3 groups and kept in single cages. During a 9 days run-in period the animals were offered ad libitum chow and water (tab water) in a standardised environment (LD cycle (3000/1500), temperature 21-23 degrees, humidity 55-65%).

At day 10 one group of animals (n=10) were put on a salt-loading diet with 2.5% NaCl in their drinking water. At day 13 another group of animals (n=10) was subsequently water deprived for 48 h. Half of these animals (n=5) were allowed access to water for 1 hr thereafter. The remaining group (n=10) was allowed water freely. All groups of animals were offered chow ad libitum during the entire period. The 4 groups of animals were decapitated in the morning of day 15 after the completion of the drinking regime and their brains removed and rapidly frozen on dry ice.

The daily intake of calories and water as well as body-weight gain/loss was monitored for all animals during the last 8 days of the experiment.

All brains were cut in a cryostat in the same fashion: 12 μm thick frontal sections through the hypothalamus were collected on Superfrost™ slides (two sections per slide). Every tenth slide was counterstained with thionin and used to located specific areas. One slide from each animal containing the PVN was processed for in situ hybridization with P33 labeled GUS3 antisense probes (in situ hybridization procedure described above). For each individual experiment all slides were processed simultaneously and exposed onto the same phosphoimager screen. The screens were scanned and the pictures analyzed on the Quantity One software (Biorad). The areas of interest (PVN (Paraventricular Hypothalamic Nucleus) and hippocampus) were delineated and the signals were quantitated as the area of GUS3 expression multiplied with the mean density of that area (with subtraction of the background defined as the mean density at the outline of the area) (Area=area×(mean minus background)=arbitrary units). The average was taken of 2 sections per animal. A one-way ANOVA with Scheffes post-hoc test was applied.

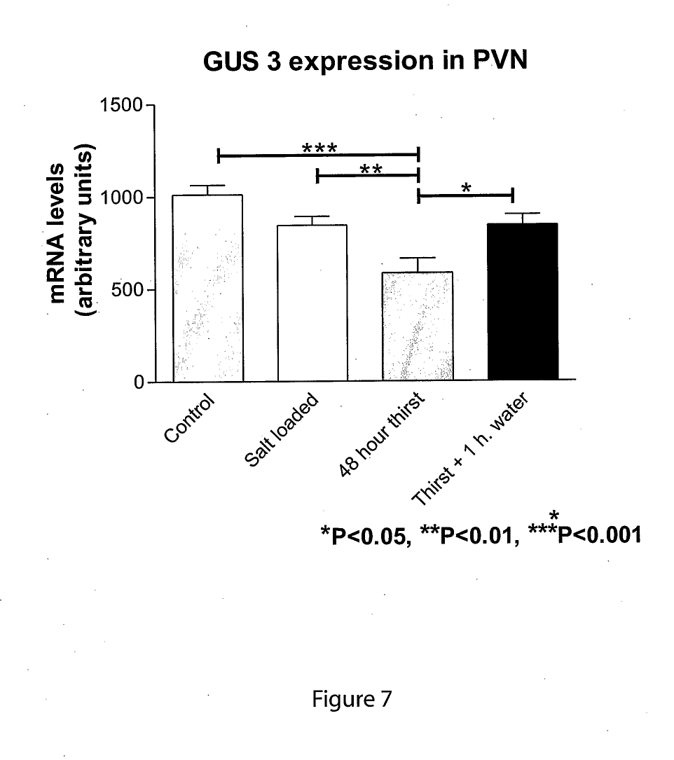

GUS3 mRNA levels in the PVN are down-regulated in animals denied access to water (P<0.001) (cf. FIG. 7) and are upregulated within one hour when access to water is regained (P<0.05). Salt-loaded animals show the same tendency as thirsted animals, albeit non-significantly. Conversely, the GUS3 mRNA levels are unaffected in the hippocampus. These results suggest that GUS3 is implicated in the regulation of water, solute, and/or metabolic homeostasis, but may also be a result of GUS3 being implicated in the regulation of a stress response.

### Example 7

### Acute and Chronic Effects of GUS3 Fragments on Food and Water Intake

### Animals and Surgery:

Thirty-six regular Sprague Dawley rats (Charles River, Germany) weighing approximately 250 gram at the start of the experiment were used for these experiments. All animals were kept under a 12/12 L/D cycle (lights on at 0300) and in temperature and humidity controlled rooms. The animals were allowed a 5 day acclimatisation period after arrival to the test facility in order to reduce stress effects.

### Study Design:

All experiments are conducted in accordance with internationally accepted principles for the care and use of laboratory animals and are approved by the Danish committee for animal research. Under Hypnorm Dormicum (Nomeco A/S, Copenhagen Denmark) anesthesia all animals were stereotaxically equipped with a stainless steel cannula aimed at the lateral ventricle (1 mm caudal and 1.5 mm lateral from bregma and 4 mm depth). During a 7 day recovery period, the rats were handled daily in order to accustom them to the experimental procedure.

Determination of the Active/most Active Peptide in a Standard 24-hour Thirst Assay

The following peptides were ordered from Schafer-N, Copenhagen (see also example 2):

GUS3 N:
ac-QFLKEGQLAAGTCEIVTLDRDSSQP
(ordered as an N-acetylated peptide due to
synthesis problems)
GUS3 M:
TIARQTARCAC
GUS3 C:
GQIAGTTRARPACVDARIIKTKQWCDMLPCLEGEGCDLLINRSGWTCTQP
GG

The animals were divided into 4 weight-matched groups prior to the test:
Group 4 Vehicle control (5 μL PBS)
Group 3 GUS3 C (10 ug) icv in 5 uL vehicle
Group 2 GUS3 M (1 ug) icv in 5 uL vehicle
Group 1 GUS3 N (10 ug) icv in 5 uL vehicle

The rats were thirsted but not food deprived twenty-four hours before the experiment. Fifteen minutes prior to reintroduction of water (time=0) the animals received an ICV injection of 5 μg of one of the 3 peptides dissolved in 5 μL vehicle or of vehicle alone. Water and food intake was recorded at time=30 minutes, 60 minutes, 2 hour, 3 hours and 24 hours.

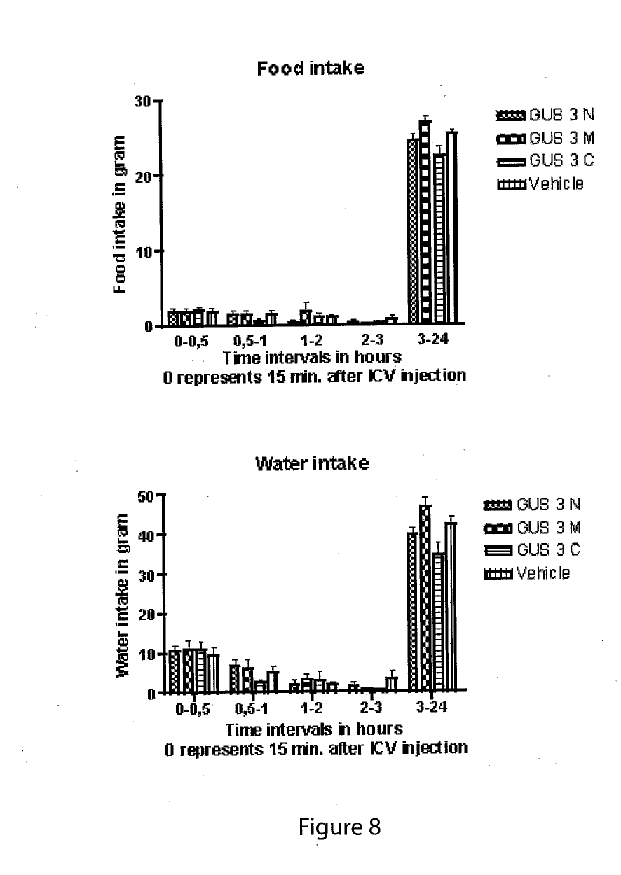

The results of the experiment are shown in FIG. 8. Intrecerebrovascular injection of the GUS3 C peptide had a statistically significant effect on food and water intake, causing both to decrease whereas the GUS3 N and GUS3 M peptides did not seem to affect these parameters. These results are consistent with a role of GUS3, notably the GUS3 C peptide in the regulation of water, electrolyte, and/or metabolism homeostasis.

The experiment is repeated with recombinant peptides produced in genetically engineered bacteria, yeast, or mammalian cell cultures.

The experiment is repeated in the acute setting with measurement of diuresis, blood pressure, heart rate etc. The measurement may be extended for several days for the measurement of long-term effects of a single dosis.

The experiment is repeated in a chronic setting where the peptides are delivered via osmotic minipumps or by repeated manual injection for several days, and an extended set of parameters is measured. This extended set of parameters include but is not limited to food intake, water intake, activity, diuresis, plasma vasopressin, blood pressure, heart rate, weight gain/loss, insulin resistance, serum free fatty acids, triglycerides, etc.

The experiment is repeated in acute and chronic settings where appropriate concentrations of the peptides are delivered intravenously to ascertain the systemic role of the peptides.

The data are evaluated by relevant statistical analyses (Statview software). Results are presented as mean±SEM (standard error of the mean). Statistical evaluation of the data is carried out using one-way analysis of variance (ANOVA) with appropriate post-hoc analysis between control and treatment groups in cases where statistical significance is established (p<0.05; Scheffe or Bonferoni).

### Example 8

### Generation of Polyclonal GUS3 Antibodies

Peptides: The Three Different GUS3 Fragments were Used for the Immunization Procedure:

GUS3 N:
ac-QFLKEGQLAAGTCEIVTLDRDSSQP
GUS3M:
TIARQTARCAC
GUS3C:
GQIAGTTRARPACVDARIIKTKQWCDMLPCLEGEGCDLLINRSGWTCTQP
GG

The peptides were coupled to bovine serum albumin (BSA fraction V; Roche Diagnostics) according to the following procedure: 1.8 mg peptide, 3.6 mg BSA, 18 mg (1-ethyl-3(3-dimethylaminopropyl))carbodiimid (Sigma), 0.6 ml N, N-dimethylformamide (Sigma) were mixed with 3.9 mL phosphate buffered saline (PBS, 50 mM) overnight. Twelve New Zealand White rabbits (Charles River, Sweden) housed under standard laboratory conditions with free access to food and water were used in the immunization experiments (4 rabbits injected with each peptide). Prior to immunization 20 mL of pre-immune blod was acquired from each rabbit. The first time rabbits were immunized with a mixture of 200 μl peptide with 300 μl Freunds complete adjuvant (Sigma). Booster injections consisted of mixes of peptide and Freunds incomplete adjuvant (Sigma). Rabbits were injected every second week and bled every second week (alternate). The blood was allowed to clot overnight at 4 degrees C., subjected to a short centrifugation, and the resulting serum frozen in aliquots at minus 20 degrees C.

### Example 9

### Immuno-staining of Rat Brain Sections

The antiserum from above is useful for confirming presence of the GUS3 peptide in vivo in support of the RT-PCR results from Example 3 that were confirming presence of GUS3 mRNA as well as the western blotting experiments in Example 9.

Twelve male SPD rats (300 g) housed under standard laboratory conditions are used for the experiments. The rats are anesthetized with 0.2 mL/100 g body weight of Hypnorm-Dormicum (1 mL contains: 0.167 mg fentanyl, 5 mg fluanisone, 2.5 mg midazolam). The rats are next vascularly perfused with heparinized KPBS (15,000 IE/L), followed] by 4% paraformaldehyde dissolved in 0.1 M phosphate buffer (pH=7.4) for 15 minutes. The brains are removed and postfixed in the same fixative over-night, cryoprotected for two days in a 30% sucrose-KPBS solution and cut in 40 μm thick frontal one-in-six series on a freezing microtome and collected in PBS.

All reactions are carried out on free-floating sections. Serum from the immunized rabbits is used diluted 1:1000 and 1:10,000. Also pre-immune serum is used as controls at the same dilutions. Immunohistochemistry is performed according to the following procedure: The sections are washed in KPBS for 3×10 minutes followed by 10 minutes incubation in 1% H₂O₂ in KPBS. Sections are then blocked for 20 minutes in 5% swine serum in KPBS containing 0.3% Triton X-100 (TX) and 1.0% bovine serum albumin (BSA). The serum is diluted in 0.3% TX and 1% BSA and sections incubated overnight at 4° C. The next day the sections are washed for 3×10 minutes in KPBS with 0.1% TX and KPBS-T before incubation for 60 minutes at room temperature in a biotinylated donkey anti-rabbit antibody (Jackson Immuno Research lab., INC.) diluted 1:2000 in KPBS-T. After another rinse for 3×10 minutes in KPBS-T followed by 60 minutes incubation in ABC-streptavidin horseradish peroxidase (Vector Elite Kit) the sections were washed for 3×10 minutes in KPBS-T before being developed in Chromagen Solution for 2-20 minutes (0.04% DAB+0.003% H₂O₂ in KPBS). The immunostaining is evaluated on a Nikon microscope and images acquired by a Nikon DCM1200 digital camera.

### Example 10

### Characterization of GUS3 Peptides by Western Blotting

### Extraction of Protein:

Rat tissue (cerebrospinal fluid, plasma, hypothalamus, and pancreas) is extracted with 500 μl solubilization buffer (200 mM Tris.Cl pH 6.8, 2% SDS, 350 mM DTT, 20 μl/mL Protease inhibitor cocktail for mammalian cells (Sigma, P8340)) per 125 mg tissue by solubilization with a rotor-stator-type blender. Samples are denatured by heating to 95 degrees for 5 min, cooled to room temperature, and treated with 5 μl benzonase (VWR, 1654) per mL at room temperature for 15 min. The lysates are cleared by centrifuging 10 minutes at 10000×g. The protein concentrations are determined by using a Bradford kit (Bio-Rad, 500-0001) as described by the manufacturer.

### Western Blotting and ECL:

Samples containing, Seeblue Plus 2 standards (Invitrogen, lanes labelled S), the synthesized peptides (GUS3 N, 50 ng, GUS3 M, 100 ng, or GUS3C, 5 ng, respectively (lanes labelled N, M, or C), 7.5 μg hypothalamic protein (lane 2), 7.5 μg pancreas protein (lane 3), 7.5 μg plasma protein (lane 4), and ˜2 μg cerebrospinal fluid protein, respectively, were run on 16.5% Criterion peptide gels (Bio-Rad) followed by blotting onto PVDF membrane as described by the manufacturer. GUS3 peptides were detected by enhanced chemoluminiscense using the following procedure: The membranes were blocked 30 minutes in StartingBlock blocking buffer (Pierce) at room temperature. The membranes were quickly washed in PBS and incubated with preimmune serum or with diluted antiserum directed against the GUS3 M, GUS3 N, and GUS3 C peptides, respectively. The Antisera were diluted 1:5000 (GUS3 N), 1:2000 (GUS3 M), 1:3000 (GUS3 C), respectively in StartingBlock and allowed to bind for 60 min. at room temp. The membranes were quickly washed in PBS followed by 6 washes (5 min. each) in PBS with 0.1% Tween. The membranes were incubated in horseradish peroxidase coupled antirabbit IgG (Do anti Rb Fab2, Jackson) diluted 1:100000 in StartingBlock for 60 min. at room temp and washed as above. Detection was performed with the Supersignal West Femto maximum sensitivity substrate (Pierce, 34095) as described by the manufacturer.

### Electrophoretic Migration of Synthetic GUS3 Fragments:

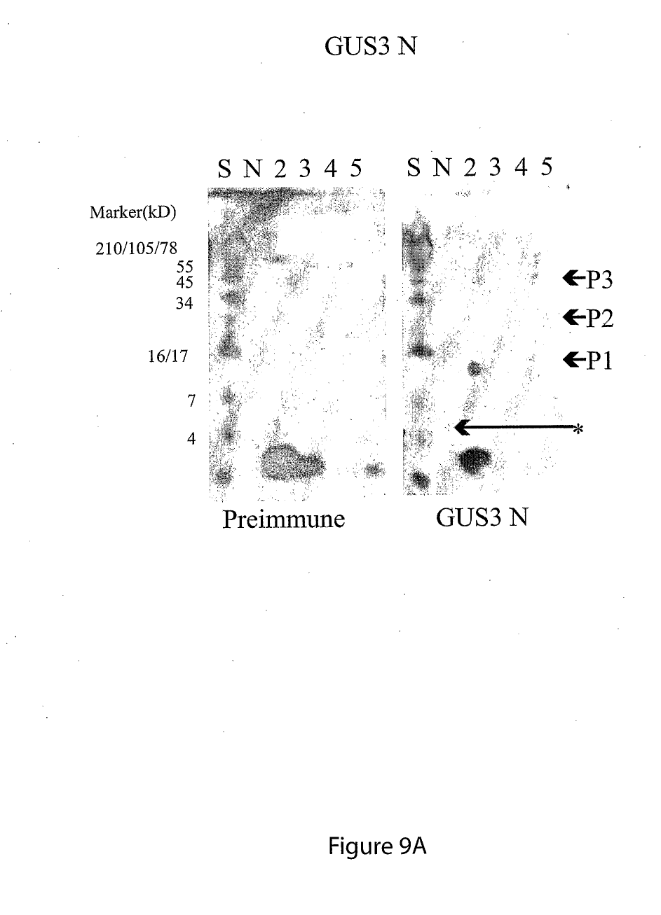

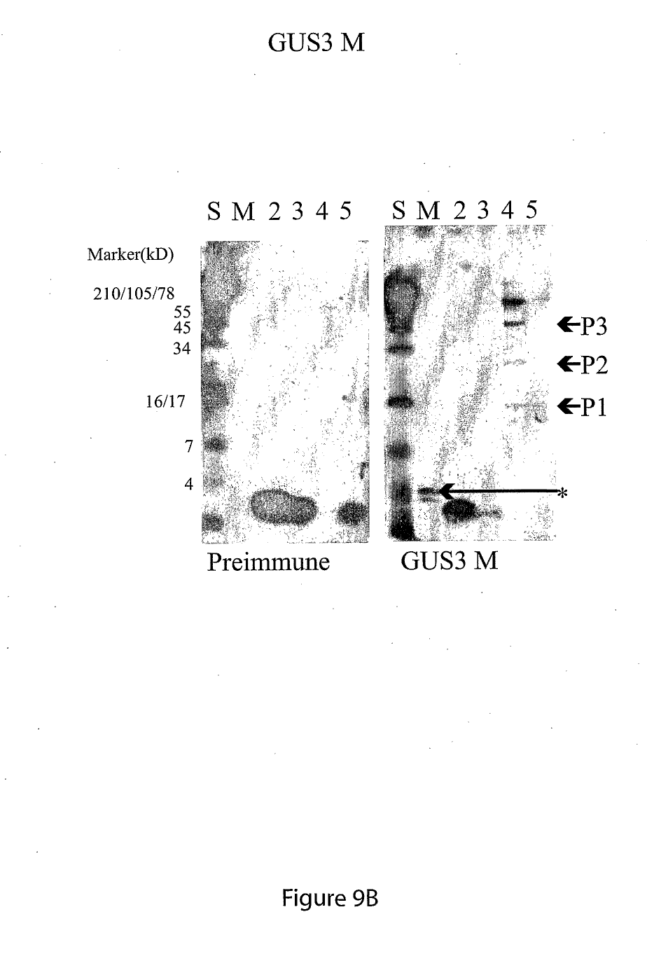

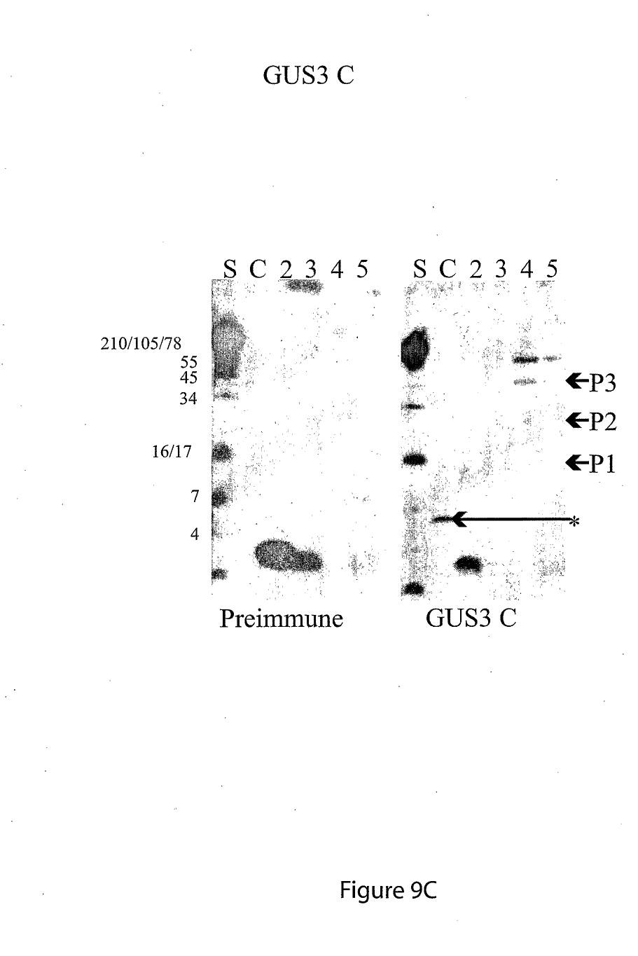

The GUS3 N and GUS3 M fragments showed an aberrant migration in the gels. The apparent molecular mass of the GUS3 C fragment is in accordance with the theoretical or calculated molecular mass (FIGS. 9A, 9B, and 9C, right panels). Thus, the apparent molecular mass of the GUS3 N fragment is 5.8 kDa (theoretical 2.7 kDa), the apparent molecular mass of the GUS3 M fragment is 4.3 kDa (Theoretical 1.2 kDa) and the apparent molecular mass of the GUS3 C fragment is 6.9 kDa (theoretical 6.5 kDa)

### GUS 3 Antisera Reactive Peptides.

A number of fragments are visible on the immunoblots (right panels on FIGS. 9A, 9B, and 9C) that are not visualized with the preimmune sera, indicating the specificity of these bands. These bands appear at the same position on all three blots, indicating that the visualized polypeptides contain at least part of all the GUS3 N, GUS3 M, and GUS3 C peptides. The bands are especially predominant in the plasma samples where bands with apparent molecular masses of 14.5 kDa (FIG. 9, band P1), 26 kDa (FIG. 9, band P2), and 46 kDa (FIG. 9, band P3). The apparent sizes of these polypeptides are not in accordance with the theoretical sizes predicted. The polypeptides may migrate abnormally on the blots as well as was seen for the synthetic fragments or to postsynthetic modifications, or, alternatively, the GUS3 encoding gene may be subjected to alternative splicing and/or alternative transcription initiation which can produce alternative transcripts and/or splice variants.

Nevertheless, the highest concentrations of GUS3 antisera binding proteins are present in the plasma samples, indicating that GUS3 is indeed a secreted molecule, possibly a hormone, produced at least in the pancreas and hypothalamus (according to the multiplex analysis) and with an effect at least partially mediated in the perifery (although the peptides injections showed that some effect could be mediated centrally.

This finding also has implications for the diagnostic value of GUS3 because monitoration of blood levels of GUS3 may be predictive for diseases affecting mammalian water, solute, and/or metabolic homeostasis.

### Example 11

### Determination of the Molecular Weight of Processed GUS 3 Peptides by Immunoprecipitation and Mass Spectrometry

Because of the unexpected migration of GUS3 peptides on acrylamide gels, it is useful to precisely determine the molecular mass of the peptides. Thus, the antisera (examples 7 and 9) that have been found to be reactive against GUS3 peptides are used for immunoprecipitation of GUS 3 peptides as described hereunder:

Extraction of Protein from Hypothalamus and Plasma

Sprague-Dawley rats are killed by decapitation, and the hypothalami dissected and isolated. Five hundred μl of lysis buffer (50 mM Tris.Cl pH 7.4, 5 mM EDTA, 1% Triton X100, 300 mM NaCl, 10 mM DTT, 25 μl protease inhibitor cocktail (Sigma) per ml) are added per 125 mg tissue and the tissue is solubilized with a rotor-stator-type blender. The lysate is incubated 5 min on ice and cleared by microcentrifuging (15 min at 16,000 g, 4° C.). The supernatant is isolated and used for immunoprecipitation.

Plasma extract is obtained by isolating blood from Sprague-Dawley rats, by adding 25 μl protease inhibitor cocktail per ml followed by the isolation of plasma addition of lysis buffer as described above followed by a 5 min incubation and a clearing by centrifugation.

### Immunoprecipitation.

One-hundred μl of Dynabeads Protein A suspension (Dynal, Sweden) are transferred to a test tube and washed twice in 0.5 ml 0.1 M Na-phosphate buffer pH 8.1.

The Dynabeads are resuspended in 90 μl 0.1 M Na-phosphate buffer pH 8.1 and 10 μl serum added. The antibodies are allowed to bind to the Dynabeads for 10 min and washed three times with 0.5 ml 0.1 M Na-phosphate buffer pH 8.1, washed twice by the addition of 1 ml 0.2 M triethanolamine, pH 8.2 and crosslinked by resuspension in 1 ml of 20 mM DMP (dimethyl pimelimidate dihydrochloride, Pierce #21666) in 0.2 M triethanolamine, pH 8.2. The suspension is incubated with rotational mixing for 30 minutes at 20° C. Then, the reaction is stopped by resuspending the Dynabeads in 1 ml of 50 mM Tris, pH 7.5 and incubating for 15 minutes with rotational mixing.

Finally, the Dynabeads are washed 3 times with 1 ml and resuspended in 200 μl of protein extract. Binding is allowed to take place for 1 hr at 2° C. for 1 hour, and the Dynabeads are washed 3 times using 1 ml PBS each time. A sample of 100 microliter (resuspended beads) is taken from the last wash to a separate tube. The beads containing the bound peptides are isolated and resuspended in sample buffer whereafter the resulting peptides are checked by Western blotting.

The remaining beads are used for laser desorption mass spectrometry, wherein the size of the bound peptides can be determined with great accuracy. The mass of the peptides are used to predict the processing of the GUS3 peptides

### Example 11

### Identification of the GUS3 Receptor by Screening of a Library

The GUS3 receptor is identified, essentially as described [23, 24]. In brief, 10⁷ Plat-E packaging cells are transiently transfected with 10 μg human brain cDNA library cloned into the pEXP1 vector (Clontech) using Lipofectamine 2000 (Invitrogen). Ba/F3 cells are infected with 1/20 diluted supernatants corresponding to an estimated multiplicity of infection of 0.3.

Subsequently, the infected cells are incubated with fluorescently labelled GUS3 peptides, and cells expressing the GUS3 receptors are isolated by sorting in a fluorescence activated cell sorter (FACS). The sorted cells are expanded in a bulk culture and reanalysed by fluorescent GUS3 peptide binding and FACS. Subsequently, the cells are sorted as above, and the sorted cells again expanded in bulk culture. A subsequent analysis by fluorescent GUS3 peptide binding and FACS shows the majority of cells being positive for GUS3 binding, and the cells are subjected to single-cell sorting and 10 subclones expanded for further analysis.

Genomic DNA is isolated from these clones; the GUS3 receptor encoding cDNA is amplified by PCR using viral vector specific primers, and the resulting cDNA cloned and sequenced.

### Example 12

### Identification of the GUS3 Receptor by a Candidate Approach

A great number of hormone and neuropeptide receptors are G-protein coupled receptors. Furthermore, a number of putative orphan G-protein receptors have been identified from genomic information available from the sequencing of the human, rat, and mouse genomes. Thus, the GUS3 receptor may also be cloned by a direct approach, where available genomic/cDNA sequences of orphan G-protein receptors are used for directional cloning of human, rat, and/or mouse putative GUS3 receptors into an expression vector.

The promiscuous G protein chimeras Galpha(16/z), 16z25 and 16z44 [25] are coexpressed with the G-protein coupled receptors and the cells subjected to activation by GUS3 peptides. Binding to the receptor and activation of the chimera is ascertained by the ability to translate GPCR activation into Ca(2+) mobilization using a fluorescence imaging plate reader (FLIPR) and aequorin.

### REFERENCES

1 Swanson, L. W. (1992) Brain maps: structure of the rat brain, Elsevier, Amsterdam
2 Krieg, W. J. S. (1932) J Comp Neurol 55, 19-89
3 Geeraedts, L. M. G., Nieuwenhuys, R. and Veening, J. G. (1990) J. Comp. Neurol. 294, 537-568
4 Geeraedts, L. M. G., Nieuwenhuys, R. and Veening, J. G. (1990) J. Comp. Neurol. 294, 507-536
5 Gustincich, S., Batalov, S., Beisel, K. W., Bono, H., Carninci, P., Fletcher, C. F., Grimmond, S., Hirokawa, N., Jarvis, E. D., Jegla, T., Kawasawa, Y., LeMieux, J., Miki, H., Raviola, E., Teasdale, R. D., Tominaga, N., Yagi, K., Zimmer, A., Hayashizaki, Y. and Okazaki, Y. (2003) Genome Res 13, 1395-401
6 Dolle, R. E. (1998) Mol Divers 4, 233-56
7 Malik, F., Delgado, C., Knusli, C., Irvine, A. E., Fisher, D. and Francis, G. E. (1992) Exp Hematol 20, 1028-35
8 Zamore, P. D. (2002) Science 296, 1265-9
9 Kohler, G. and Milstein, C. (1975) Nature 256, 495-7
10 Cote, R. J., Morrissey, D. M., Houghton, A. N., Beattie, E. J., Jr., Oettgen, H. F. and Old, L. J. (1983) Proc Natl Acad Sci USA 80, 2026-30
11 Huse, W. D., Sastry, L., Iverson, S. A., Kang, A. S., Alting-Mees, M., Burton, D. R., Benkovic, S. J. and Lerner, R. A. (1989) Science 246, 1275-81
12 Pearson, W. R. and Lipman, D. J. (1988) Proc Natl Acad Sci USA 85, 2444-8
13 Jensen, L. J., Gupta, R., Staerfeldt, H. H. and Brunak, S. (2003) Bioinformatics 19, 635-42
14 Jensen, L. J., Ussery, D. W. and Brunak, S. (2003) Genome Res 13, 2444-9
15 Karolchik, D., Baertsch, R., Diekhans, M., Furey, T. S., Hinrichs, A., Lu, Y. T., Roskin, K. M., Schwartz, M., Sugnet, C. W., Thomas, D. J., Weber, R. J., Haussler, D. and Kent, W. J. (2003) Nucleic Acids Res 31, 51-4
16 Kent, W. J., Sugnet, C. W., Furey, T. S., Roskin, K. M., Pringle, T. H., Zahler, A. M. and Haussler, D. (2002) Genome Res 12, 996-1006
17 Kent, W. J. (2002) Genome Res 12, 656-64
18 Boon, K., Osorio, E. C., Greenhut, S. F., Schaefer, C. F., Shoemaker, J., Polyak, K., Morin, P. J., Buetow, K. H., Strausberg, R. L., De Souza, S. J. and Riggins, G. J. (2002) Proc Natl Acad Sci USA 99, 11287-11292.
19 Nielsen, H., Engelbrecht, J., Brunak, S, and von Heijne, G. (1997) Protein Eng 10, 1-6
20 Nielsen, H. and Krogh, A. (1998) Proc Int Conf Intell Syst Mol Biol 6, 122-30
21 Higgins, D. G. and Sharp, P. M. (1988) Gene 73, 237-44
22 Jensen, J., Serup, P., Karlsen, C., Nielsen, T. F. and Madsen, 0. D. (1996) J Biol Chem 271, 18749-58
23 Kitamura, T., Onishi, M., Kinoshita, S., Shibuya, A., Miyajima, A. and Nolan, G. P. (1995) Proc Natl Acad Sci USA 92, 9146-50
24 Yamauchi, T., Kamon, J., Ito, Y., Tsuchida, A., Yokomizo, T., Kita, S., Sugiyama, T., Miyagishi, M., Hara, K., Tsunoda, M., Murakami, K., Ohteki, T., Uchida, S., Takekawa, S., Waki, H., Tsuno, N. H., Shibata, Y., Terauchi, Y., Froguel, P., To be, K., Koyasu, S., Taira, K., Kitamura, T., Shimizu, T., Nagai, R. and Kadowaki, T. (2003) Nature 423, 762-9
25 Liu, A. M., Ho, M. K., Wong, C. S., Chan, J. H., Pau, A. H. and Wong, Y. H. (2003) J Biomol Screen 8, 39-49

1. Use in a medicament of at least one compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; a functional variant of any of these peptides; a modulator of any of these peptides; a peptide that binds polyclonal antibodies raised against any of these peptides; a DNA sequence encoding any of these peptides; and an antisense-polynucleotide to a nucleotide sequence encoding any of these peptides.

2. Use in a medicament of antibodies that are specific to at least one compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; a functional variant of any of these peptides; and a modulator of any of these peptides.

3. Use of at least one compound in a medicament for regulating thirst, wherein said at least one compound is selected from the group consisting of the compounds of claim 1 and antibodies of claim 2.

4. Use of at least one compound in a medicament for regulating appetite, wherein said at least one compound is selected from the group consisting of the compounds of claim 1 and antibodies of claim 2.

5. Use of at least one compound in a medicament for regulating water and/or solute balance, wherein said at least one compound is selected from the group consisting of the compounds of claim 1 and antibodies of claim 2.

6. Use of at least one compound for manufacturing a medicament for preventing, treating, or regulating a hypothalamic function and/or disorder in an animal, wherein said at least one compound is selected from the group consisting of the compounds of claim 1 and antibodies of claim 2.

7. Use of at least one compound for manufacturing a medicament suitable for treatment of water and solute imbalances, wherein said at least one compound is selected from the group consisting of the compounds of claim 1 and antibodies of claim 2.

8. Use of at least one compound for manufacturing a medicament suitable for regulating thirst, wherein said at least one compound is selected from the group consisting of the compounds of claim 1 and antibodies of claim 2.

9. Use of at least one compound for manufacturing a medicament suitable for regulating appetite, wherein said at least one compound is selected from the group consisting of the compounds of claim 1 and antibodies of claim 2.

10. Use according to claim 1, wherein said compound is selected from the group consisting of a peptide with the sequence defined in SEQ ID NO 2, a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides.

11. Use according to claim 1, wherein said compound is selected from the group consisting of a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides.

12. Use according to claim 1, wherein said compound is GUS3C or a functional variant thereof.

13. A method of preventing, treating or regulating a hypothalamic function and/or disorder in an animal, comprising administering to the animal an effective amount of at least one active compound selected from: SEQ ID NO 2; residues 66-125 from SEQ ID NO 2; residues 53-63 from SEQ ID NO 2; residues 26-50 from SEQ ID NO 2; a functional variant of any of these peptides; a peptide that binds polyclonal antibodies raised against any of these peptides; antibodies specific to at least one of these peptides, a DNA sequence encoding any of these peptides; and an anti-sense nucleotide to a nucleotide sequence encoding any of these peptides.

14. A method according to claim 13 wherein the hypothalamic disorder is selected from: malfunctions in water and electrolyte homeostasis; energy homeostasis; insulin resistance; dyslipidaemia; arterial blood pressure regulation; dysfunction of thirst regulation; and dysfunction of appetite regulation.

15. A method according to claim 13 wherein the hypothalamic disorder is a dysfunction that leads to an abnormal body weight regulation, eventually leading to type II diabetes and the metabolic syndrome X.

16. A pharmaceutical composition for regulating a hypothalamic function comprising at least one compound according to claim 1 or antibodies according to claim 2 as well as pharmaceutically acceptable ingredients.

17. A pharmaceutical composition for regulating a hypothalamic function comprising a siRNA polynucleotide specific for one of the following: a polynucleotide encoding peptide with the sequence defined in SEQ ID NO 2; a polynucleotide encoding a peptide with the sequence defined by residues 66-125 from SEQ ID NO 2; a polynucleotide encoding a peptide with the sequence defined by residues 53-63 from SEQ ID NO 2; a polynucleotide encoding a peptide with the sequence defined by residues 26-50 from SEQ ID NO 2; a peptide that binds polyclonal antibodies raised against any of these peptides; a DNA sequence encoding any of these peptides; and a polynucleotide encoding a functional variant of any of these peptides.

18. A method of identifying an interaction partner to GUS3 comprising using at least one compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; a peptide that binds polyclonal antibodies raised against any of these peptides; a peptide that binds polyclonal antibodies raised against any of these peptides; a functional variant of any of these peptides; and a DNA sequence encoding any of these peptides;
to screen an expression library for interaction partners.

19. A method of identifying a modulator of GUS3 comprising using a compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2, a functional variant of any of these peptides, and a peptide that binds polyclonal antibodies raised against any of these peptides; to screen an array of compounds for binding partners and subsequently determining the effect of this binding upon the biological activity of GUS3.

20. A method of diagnosing or prognosticating a metabolic disorder in an animal comprising determining the sequence of the polynucleotide, which encodes GUS3, and comparing the sequence with SEQ ID NO: 1 to identify differences in the sequence and using this information for diagnostic and/or prognostic purposes.

21. A method of diagnosing or prognosticating a metabolic disorder in an animal comprising determining the level of GUS3 in a biological sample using an antibody of claim 2, and using the measurement to evaluate the state of the animal.

22. A method of effecting a change in water and/or food intake in an animal comprising administering to the animal an effective amount of a compound selected from: a peptide with the sequence defined in SEQ ID NO 2; a peptide with the sequence defined by residues 66-125 from SEQ ID NO 2; a peptide with the sequence defined by residues 53-63 from SEQ ID NO 2; a peptide with the sequence defined by residues 26-50 from SEQ ID NO 2; a functional variant of any of these peptides; a peptide that binds polyclonal antibodies raised against any of these peptides; and a DNA sequence encoding any of these peptides.

23. Use according to claim 2, wherein said compound is selected from the group consisting of a peptide with the sequence defined in SEQ ID NO 2, a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides.

24. Use according to claim 2, wherein said compound is selected from the group consisting of a peptide with the sequence defined by residues 66-125 from SEQ ID NO 2, a peptide with the sequence defined by residues 53-63 from SEQ ID NO 2, a peptide with the sequence defined by residues 26-50 from SEQ ID NO 2, and a functional variant of any of these peptides. 12. Use according to any one of claims 1-9, wherein said compound is GUS3C or a functional variant thereof.

25. Use according to claim 2, wherein said compound is GUS3C or a functional variant thereof.
